(19) United States
(12) Patent Application Publication
(10) Pub. No.: US 2002/0164346 A1
(43) Pub. Date: Nov. 7, 2002

(54) Altered peptide ligands

(76) Inventor: Charles A. Nicolette, Framingham, MA (US)

Correspondence Address: GENZYME CORPORATION C/O MCCUTCHEN, DOYLE, BROWN,, & ENERSEN, MCCUTCHEN, DOYLE, BROWN & ENERSEN, LLP, THREE EMBARCADERO CENTER, SAN FRANCISCO, CA, 94111, US

(21) Appl. No.: 10/077,629

(22) Filed: Feb. 14, 2002

Related U.S. Application Data
(60) Provisional application No. 60/269,077, filed on Feb. 14, 2001.

(51) Int. Cl. A61K 39/00

(52) U.S. Cl. 424/185.1

(57) ABSTRACT

The present invention provides compositions comprising altered peptide ligands that elicit immune responses in a subject to a native peptide. This invention also provides methods to raise T cell populations as well as a substantially purified population of said T cells. Altered peptide ligands find application in a wide variety of immunomodulatory protocols, including methods to induce or increase an immune response, as well as in methods to suppress or reduce an undesirable immune response, to a corresponding natural epitope.

### CROSS-REFERENCE TO RELATED APPLICATIONS

This application claims the benefit under 35 U.S.C. § 119(e) of U.S. Provisional patent application No. 60/269,077, filed Feb. 14, 2001, the content of which is hereby incorporated by reference into the present disclosure.

### TECHNICAL FIELD

This invention relates to the field of antigenic epitopes, and more particularly to altered peptide ligands and methods of using these ligands to stimulate distinct populations of T cells having different T cell receptor Vβ recombinations.

### BACKGROUND

The recognition of antigenic determinants, also known as epitopes, presented by molecules of the Major Histocompatibility Complex (MHC) plays a central role in the establishment, maintenance and execution of mammalian immune responses. T cell recognition of epitopes presented by cell surface MHC molecules expressed by somatic cells and antigen presenting leukocytes function to control invasion by infectious organisms such as viruses, bacteria, and parasites. In addition, it has been demonstrated that cytotoxic T lymphocytes (CTLs) can specifically recognize certain cancer cell antigens and lyse tumor cells expressing these antigens. Furthermore, inappropriate T cell activation has been shown to play a central role in a certain debilitating autoimmune diseases such as, for example, rheumatoid arthritis, multiple sclerosis, and asthma. Thus, T cell recognition of antigenic epitopes presented by MHC molecules play a central role in mediating immune responses in multiple pathological conditions.

Immunotherapeutic strategies have been developed that attempt to “modulate” various aspects of the immune response associated with a pathological condition. Many of these approaches depend in part upon the use of identified and characterized tumor-specific antigens.

In one strategic area, manipulation of antibody molecules and humoral immune responses directed against normal or mutated cellular antigens expressed in cancers or virally infected cells provide therapeutic and diagnostic agents. Vaccines can produce antibodies directed against tumor specific antigens for immunotherapy to produce antibody-dependent cellular cytotoxicity, complement-dependent cytolysis, and apoptosis (Sinkovics and Horvath (2000) Int. J. Oncol. 16(1):81-96; Weiner (1999) Semin. Oncol. 26:43-51). Antibody immuno-conjugates derived from tumor antigen specific monoclonal antibodies are useful as delivery agents for cytotoxic agents and radionuclides or as imaging agents for diagnostic applications (Roselli et al. (1996) Anticancer Res. 16(4B):2187-2192; Trail and Bianchi (1999) 11(5):584-588). Anti-tumor antibodies to induce anti-idiotype antibodies that mimic the characteristics of tumor antigens and which are capable of further inducing anti-tumor humoral and cellular immune responses against tumors (Fagerberg et al. (1995) 92(11):4773-4777).

In another strategic area, antigens have been widely used for the purposes of vaccination against pathogens, induction of an immune response to a cancerous cell, reduction of an allergic response, reduction of an immune response to a self-antigen occurring as a result of an autoimmune disorder, reduction of allograft rejection, and induction of an immune response to a self antigen for the purpose of contraception.

In cancer, tumor-specific T cells can be derived from patients which are capable of binding and lysing tumor cells that display the corresponding tumor-associated antigen on their cell surfaces. Tumor-specific T cells are localized at several sites within cancer patients, including in the blood (where they can be found in the peripheral and mononuclear cell fractions), in primary and secondary lymphoid tissue, e.g., the spleen, in ascites fluid in ovarian cancer patients (tumor-associated lymphocytes or “TALs”) or within the tumor itself (tumor-infiltrating lymphocytes or “TILs”). Of these T cells populations, TILs have been the most useful in the identification of tumor antigens and epitopes thereof.

The specificity of tumor-specific T cells is based on the ability of the T cell receptor (TCR) to recognize and bind to a short amino acid sequence which is presented on the surface of the tumor cells by MHC class I and, in some cell types, class II molecules. In brief, these amino acid binding sequences (also termed “ligands” or “epitopes”) are derived from the proteolytic degradation of intracellular proteins encoded by genes that are either uniquely or aberrantly expressed in tumor or cancer cells. The peptide ligands and intracellular proteins containing these epitopes are designated “tumor antigens”.

The availability of tumor-specific T cells has facilitated the identification of some tumor antigens which have been employed in cancer vaccine compositions with varying degrees of success. Attempts to provoke anti-tumor responses in vivo by vaccination with protein or peptide fragments, however, are often unsuccessful, presumably because the protein or peptide fragments fail to access the cytosol of a cell and, therefore, are not properly processed and presented to effector cells. Conventional methods for culturing and subcloning of tumor-specific T cells are known in the art. Once a potent anti-tumor T cell population is recovered, it can be used to identify tumor antigens via conventional, but often tedious, expression cloning methodology. Kawakami Y. et al. (1994) Proc. Natl. Acad. Sci. USA 91(9):3515-3519. The results of numerous attempts to use expression cloning for generating tumor-specific T cells in vitro, however, suggest that this methodology is unreliable.

In a different approach, requiring a known pathogen- or tumor-related antigen, methods that attempt to identify the native epitope have been developed. For example, putative epitopes can be predicted using a computer to scan the sequence of the gene (antigen) for amino acid sequences that contain a “motif” or a defined pattern of amino acid residues associated with a particular HLA allele. See, e.g., Englehard, V. H., (1994) Annu. Rev. Immunol. 12:181; Rammenesee, H. et al. (1993) Annu. Rev. Immunol. 11:213. The “predicted” epitope sequences can then be synthesized and tested. Although many epitope sequences have been “predicted” from scanning full-length protein sequences by “motif”, upon testing in standard functional assays, the vast majority of these “predicted” epitopes fail to be immunogenic. Other techniques include, for example, peptide elution followed by database searching (Hunt, D. F. et al., (1992) Science 255:1261; Udaka, K. et al., (1992) Cell 69:989); isolation and identification of the antigen from complex antigen mixtures (Van de Wal, Y. et al., (1998) Proc. Natl. Acad. Sci. USA 95:10050; Lamb, J. et al., (1987) Immunology 60:1); screening expression libraries and subsequent database searches (Boon, T., et al., (1994) Annu. Rev. Immunol. 12:337; Neophytou, P. I. et al., (1996) Proc. Natl. Acad. Sci. USA 93:2014; Gavin, M. A. et al., (1994) Eur. J. Immunol. 24:2124); peptide positional scanning of combinatorial libraries (Gundlach, B. J. et al., (1996) J. Immunol. Meth. 192:149; Blake, J. et al., (1996) J. Exp. Med. 184:121; Hiemstra, H. S. et al., (1997) Proc. Natl. Acad. Sci. USA 94:10313; Hemmer, B. et al., (1997) J. Exp. Med. 185:165 1), and the like.

More recently, combinatorial peptide and non-peptide chemistry methodologies have provided additional tools for determining T cell epitopes. Epitopes so determined typically “mimic” the native epitope in that they bear a definable sequence similarity thereto (e.g., conservative substitutions as well as identical amino acids), but not necessarily absolute identity therewith. Epitope mimics can be designed by directly modifying the sequence of known epitopes or defined de novo with randomized molecular libraries followed by database searching to identify the native antigen. (Gavin, M. A. et al. (1994) Eur. J. Immunol. 24:2124; Blake, H., et al. (1996) J. Exp. Med. 184:121; Chen, Y. Z. et al., (1996) J. Immunol. 157:3783; Strausbauch, M. A. et al., (1998) Intl. Immunol. 10:421).

While it is often possible to identify the T cell epitope of a known antigenic polypeptide, the same does not hold true for the identification of novel native antigens and epitopes. The application of conventional methodology has been insufficient to address and/or overcome the considerable complexities and variables associated with identification of novel antigens and/or epitopes thereof. Such issues continue to present a challenge to skilled artisans in the field of the invention.

Intriguingly, it has also been shown that it is possible to improve the effectiveness of natural epitopes by introducing single or multiple amino acids substitutions that alter their sequence (Valmori et al. (2000) J. Immunol 164(2):1125-1131). This suggests that the generation of immunogenic altered epitopes is of great interest and promise for the treatment of a variety of indications, including cancer.

Thus, a need exists for additional, therapeutically effective vaccines. This invention satisfies this need and provides related advantages as well.

### DESCRIPTION OF THE DISCLOSURE

This invention provides a method to select altered peptide species for administration to a subject presenting a native ligand, e.g., a tumor or viral antigen for the purpose of activating an immune response against the native ligand is provided. The altered peptide species are designed and selected to active an immune response (T cell or B cell) against a native or cognate ligand.

More than one or a plurality of altered peptide species are manufacture and screened for the ability to active an immune response. The population of altered peptides are then further assayed for the ability to activate populations of T cells, wherein at least two members of the population raise T cells with distinct T cell receptor Vβ recombinations. In one aspect, the altered peptides are selected based on the ability of each to activate different T cell clones from each other. In a further aspect, the altered peptides are selected based on the ability to activate a different subpopulation of CTLs.

Various combinations of peptides can be selected. For example, at least two altered peptides, or at least three, or at least 2 to 6 altered peptides are selected.

The altered peptides are selected and can be combined in a carrier, e.g., a pharmaceutically acceptable carrier for administration to a subject. Alternatively, the peptides are present in a host cell. The host cell can be combined with a carrier, e.g., a pharmaceutically acceptable carrier.

Polynucleotides encoding the peptides are further provided, alone or in combination with a carrier, e.g., a pharmaceutically acceptable carrier. Vectors and host cells containing the polynucleotides are yet further provided by this invention. Vectors and host cells can be combined with a carrier such as a pharmaceutically acceptable carrier.

The compositions of this invention are useful to modulate an immune response in a subject. In another aspect, they are useful to educate naïve immune effector cells. The combination of immune effector cell populations are further provided by this invention. In one aspect, they are combined with a carrier, e.g.,a pharmaceutically acceptable carrier.

This invention also provides administration of the compositions to a subject to activate or induce an immune response against the native ligand.

### DESCRIPTION OF THE FIGURES

FIG. 1 shows reactivity of altered peptides in a ⁵¹Cr-release assay.

FIG. 2 shows the results of the peptides identified in FIG. 1 when screened in a further ⁵¹Cr-release assay.

FIG. 3 shows that native ligand is relatively poor at eliciting reactive T cells while the altered peptides did elicit reactive T cell.

FIG. 4 shows the results of an assay determining specificity of T cells educated with altered peptides of this invention.

In all figures “SP” indicates a selected altered peptide, e.g., SP1 is altered peptide #1.

### MODES FOR CARRYING OUT THE INVENTION

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual,” second edition (Sambrook et al., 1989); “Oligonucleotide Synthesis” (M. J. Gait, ed., 1984); “Animal Cell Culture” (R. I. Freshney, ed., 1987); the series “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987, and periodic updates); “PCR: The Polymerase Chain Reaction,” (Mullis et al., eds., 1994); “Current Protocols in Immunology” (J. E. Coligan et al., eds., 1991).

Definitions

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

“Immune response” broadly refers to the antigen-specific responses of lymphocytes to foreign substances. Any substance that can elicit an immune response is said to be “immunogenic” and is referred to as an “immunogen”. All immunogens are antigens, however, not all antigens are immunogenic. An immune response of this invention can be humoral or cell-mediated.

The term “ligand” as used herein refers to any molecule that binds to a specific site (i.e., ‘ligand site’) on another molecule. For example, a ligand may, in one embodiment, confer specificity of a protein in a reaction with an immune effector cell. It is the ligand site (i.e., epitope, determinant) within the protein that combines directly with the complementary binding site (i.e., receptor) on the immune effector cell.

In a preferred embodiment, a ligand of the invention binds to an antigenic determinant or epitope on an immune effector cell, such as a B cell receptor (BCR) or a T cell receptor (TCR). A ligand may be an antigen, peptide, protein or epitope of the invention. In one embodiment, invention ligands bind to a receptor on a B cell. In one embodiment, the ligand of the invention is about 4 to about 8 amino acids in length.

In a further embodiment, invention ligands bind to an MHC class I molecule. In one embodiment, the ligand of the invention is about 7 to about 11 amino acids in length.

In a yet further embodiment, invention ligands bind to an MHC class II molecule. In one embodiment, the ligand of the invention is about 10 to about 20 amino acids long.

In most aspects described herein, a “ligand” is an antigen or an epitopic fragment of the antigen. Examples of which include, but are not limited to, tumor antigens and viral antigens.

A “native” or “cognate” or “wild-type” ligand is a polypeptide which contains a ligand site, which has been isolated from a natural biological source, and which may or may not be recognized by the immune system.

An “altered peptide” is a ligand having a primary sequence that is different from that of the corresponding cognate native or “wild-type” ligand. Altered ligands, also referred to as non-native-, modified- and/or, synthetic ligands or peptides or proteins or genes encoding them. Altered peptides can be made by various means, including but not limited to, synthetic or recombinant methods and include, but are not limited to, antigenic peptides that are differentially modified during or after translation, e.g., by phosphorylation, glycosylation, cross-linking, acylation, proteolytic cleavage, linkage to an antibody molecule, membrane molecule or other ligand. (Ferguson et al. (1988) Ann. Rev. Biochem. 57:285-320).

The term “tumor-associated antigen” or “TAA” refers to an antigenic peptide that is associated with or specific to a tumor and is presented to T cells by MHC molecules. Examples of known TAAs include gp100, MART and MAGE.

The terms “major histocompatibility complex” or “MHC” refers to a complex of genes encoding cell-surface molecules that are required for antigen presentation to T cells and for rapid graft rejection. In humans, the MHC is also known as the “human leukocyte antigen” or “HLA” complex. The proteins encoded by the MHC are known as “MHC molecules” and are classified into class I and class II MHC molecules. Class I MHC includes membrane heterodimeric proteins made up of an cc chain encoded in the MHC noncovalently linked with the β2-microglobulin. Class I MHC molecules are expressed by nearly all nucleated cells and have been shown to function in antigen presentation to CD8⁺ T cells. Class I molecules include HLA-A, B, and C in humans. Class II MHC molecules also include membrane heterodimeric proteins consisting of noncovalently associated α and β chains. Class II MHC molecules are known to function in CD4⁺ T cells and, in humans, include HLA-DP, -DQ, and DR. In a preferred embodiment, invention compositions and ligands can complex with MHC molecules of any HLA type. Those of skill in the art are familiar with the serotypes and genotypes of the HLA. See: bimas.dcrt.nih.gov/cgi-bin/molbio/hla coefficient viewing page. Rammensee, H. G., Bachmann, J., and Stevanovic, S. MHC Ligands and Peptide Motifs (1997) Chapman & Hall Publishers; Schreuder, G. M. Th., et al., The HLA Dictionary (1999) Tissue Antigens 54:409-437.

The term “antigen-presenting matrix”, as used herein, intends a molecule or molecules which can present antigen in such a way that the antigen can be bound by a T cell antigen receptor on the surface of a T cell. An antigen-presenting matrix can be on the surface of an antigen-presenting cell (APC), on a vesicle preparation of an APC, or can be in the form of a synthetic matrix on a solid support such as a bead or a plate. An example of a synthetic antigen-presenting matrix is purified MHC class I molecules complexed to β2-microglobulin, multimers of such purified MHC class I molecules, purified MHC Class II molecules, or functional portions thereof, attached to a solid support.

The term “antigen presenting cells (APCs)” refers to a class of cells capable of presenting or processing one or more antigens and displaying fragments thereof in the form of a peptide-MHC complex on the cell surface together with costimulatory molecules required for lymphocyte activation. While many types of cells may be capable of presenting antigens on their cell surface for T cell recognition, only professional APCs have the capacity to present antigens in an efficient amount and further to activate T cells and initiate the cytolytic T cell response against the antigen. APCs can be intact whole cells such as macrophages, B-cells and dendritic cells (DCs); or other molecules, naturally occurring or synthetic, such as purified MHC class I molecules complexed to β2-microglobulin.

The term “dendritic cells (DCs)” refers to a diverse population of morphologically similar cell types found in a variety of lymphoid and non-lymphoid tissues (Steinman (1991) Ann. Rev. Immunol. 9:271-296). Dendritic cells constitute the most potent and preferred APCs in the organism. A subset, if not all, of dendritic cells are derived from bone marrow progenitor cells, circulate in small numbers in the peripheral blood and appear either as immature Langerhans' cells or terminally differentiated mature cells. While the dendritic cells can be differentiated from monocytes, they possess distinct phenotypes. For example, a particular differentiating marker, CD14 antigen, is not found in dendritic cells but is possessed by monocytes. Also, mature dendritic cells are not phagocytic, whereas the monocytes are strongly phagocytosing cells. It has been shown that DCs provide all the signals necessary for T cell activation and proliferation.

The term “antigen presenting cell recruitment factors” or “APC recruitment factors” include both intact, whole cells as well as other molecules that are capable of recruiting antigen presenting cells. Examples of suitable APC recruitment factors include molecules such as interleukin 4 (IL-4), granulocyte macrophage colony stimulating factor (GM-CSF), Sepragel and macrophage inflammatory protein 3 alpha (MIP3α). These are available from Immunex, Schering-Plough, Genzyme, and R&D Systems (Minneapolis, Minn.). They also can be recombinantly produced using the methods disclosed in Current Protocols In Molecular Biology (F. M. Ausubel et al., eds. (1987)). Peptides, proteins and compounds having the same biological activity as the above-noted factors are included within the scope of this invention.

The term “immune effector cells” refers to cells capable of binding a ligand, e.g., an antigen and which thereby mediate an immune response. These cells include, but are not limited to, T cells, B cells, monocytes, macrophages, NK cells and cytotoxic T lymphocytes (CTLs), for example CTL lines, CTL clones, and CTLs from tumor, inflammatory, or other infiltrates. Productive engagement of T cell receptors (TCRs) by MHC: ligand complexes leads to T cell proliferation and differentiation of their progeny into armed effector T cells. The armed effector T cell progeny can then act on any target cell that displays that particular antigen on its surface. Effector T cells can mediate a variety of functions. One set of important functions is the killing of infected cells by CD8⁺ CTLs and the activation of macrophages by T_{H}1 cells, which together make up cell-mediated immunity. Another function of effector T cells is the activation of B cells by both T_{H}2 and T_{H}1 cells to produce different types of antibody, thus driving the humoral immune response.

The term “immune effector molecule” as used herein, refers to molecules capable of antigen-specific binding, and includes antibodies, T cell antigen receptors, and MHC class I and class II molecules.

A “naïve” immune effector cell is an immune effector cell that has never been exposed to an antigen capable of activating that cell. Activation of naïve immune effector cells requires both recognition of the peptide:MHC complex on a professional APC and the simultaneous delivery of a costimulatory signal by the APC in order to proliferate and differentiate into antigen-specific armed effector T cells.

As used herein, the term “educated, antigen-specific immune effector cell”, is an immune effector cell as defined above, which has previously encountered a specific antigen. In contrast with its naive counterpart, activation of an educated, antigen-specific immune effector cell does not require a costimulatory signalrecognition of the peptide:MHC complex is sufficient for activation.

“Activated”, when used in reference to a T cell, implies that the cell is no longer in G₀ phase, and begins to produce one or more of cytotoxins, cytokines, and other related membrane-associated proteins characteristic of the cell type (e.g., CD8⁺ or CD4⁺), is capable of recognizing and binding any target cell that displays the particular antigen on its surface, and releasing its effector molecules.

In the context of the present invention, the term “recognized” refers to the productive engagement of the epitope or antigenic determinant of the immune effector cell by a ligand which initiates the immune response. The term “cross-reactive” is used to describe altered ligands of the invention having certain properties which are functionally overlapping or convergent with the cognate native ligand. More particularly, the immunogenic properties of a native ligand and/or native ligand-specific immune effector cells are shared, to a certain extent, by the altered ligands such that the altered ligands and/or altered ligand-specific immune effector cell populations are “cross-reactive” therewith. For purposes of this invention, cross-reactivity is manifested at multiple levels: (i) at the T cell level, i.e., altered ligands of the invention bind the TCR of and activate T cells having ‘ligand-specific’ effector functions and which additionally can effectively target and lyse cells displaying the native ligand; and (ii) at the antibody level, e.g., “anti”-altered ligand antibodies can detect, recognize and bind the native ligand and initiate effector mechanisms in an immune response which ultimately result in elimination of the native ligand from the host.

As used herein, the term “inducing an immune response in a subject” is a term well understood in the art and intends that an increase of at least about 2-fold, more preferably at least about 5-fold, more preferably at least about 10-fold, more preferably at least about 100-fold, even more preferably at least about 500-fold, even more preferably at least about 1000-fold or more in an immune response to target ligand can be detected or measured, after introducing the target ligand into the subject, relative to the immune response (if any) before introduction of the target ligand into the subject. An immune response to target ligand includes, but is not limited to, production of a ligand-specific (or epitope-specific) antibody, and production of an immune cell expressing on its surface a molecule which specifically binds to a target ligand.

“Co-stimulatory molecules” are involved in the interaction between receptor-ligand pairs expressed on the surface of antigen presenting cells and effector cells. Research accumulated over the past several years has demonstrated convincingly that resting T cells require at least two signals for induction of cytokine gene expression and proliferation (Schwartz R. H. (1990) Science 248:1349-1356 and Jenkins M. K. (1992) Immunol. Today 13:69-73). One signal, the one that confers specificity, can be produced by interaction of the TCR/CD3 complex with an appropriate MHC/peptide complex. The second signal is not antigen specific and is termed the “co-stimulatory” signal. This signal was originally defined as an activity provided by bone-marrow-derived accessory cells such as macrophages and dendritic cells, the so called “professional” APCs. Several molecules have been shown to enhance co-stimulatory activity. These are heat stable antigen (HSA) (Liu Y. et al. (1992) J. Exp. Med. 175:437-445), chondroitin sulfate-modified MHC invariant chain (Ii-CS) (Naujokas M. F. et al. (1993) Cell 74:257-268), intracellular adhesion molecule 1 (ICAM-1) (Van Seventer G. A. (1990) J. Immunol. 144:4579-4586), B7-1, and B7-2/B70 (Schwartz R. H. (1992) Cell 71:1065-1068). These molecules each appear to assist co-stimulation by interacting with their target ligands on the T cells. Co-stimulatory molecules mediate co-stimulatory signal(s), which are necessary, under normal physiological conditions, to achieve fall activation of naïve T cells. One exemplary receptor-ligand pair is the B7 co-stimulatory molecule on the surface of APCs and its counter-receptor CD28 or CTLA-4 on T cells (Freeman et al. (1993) Science 262:909-911; Young et al. (1992) J. Clin. Invest. 90:229; and Nabavi et al. (1992) Nature 360:266-268). Other important co-stimulatory molecules are CD40, CD54, CD80, and CD86. The term “co-stimulatory molecule” encompasses any single molecule or combination of molecules which, when acting together with a peptide/MHC complex bound by a TCR on the surface of a T cell, provides a co-stimulatory effect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with peptide/MHC complex, binds to a cognate ligand and results in activation of the T cell when the TCR on the surface of the T cell specifically binds the peptide. Co-stimulatory molecules are commercially available from a variety of sources, including, for example, Beckman Coulter, Inc. (Fullerton, Calif.). It is intended, although not always explicitly stated, that molecules having similar biological activity as wild-type or purified co-stimulatory molecules (e.g., recombinantly produced or muteins thereof) are intended to be used within the spirit and scope of the invention.

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, California).

The term “modulate an immune response” includes inducing (increasing, eliciting) an immune response; and reducing (suppressing) an immune response. An immunomodulatory method (or protocol) is one that modulates an immune response in a subject.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1α, interleukin-11 (IL-11), MIP-11, leukemia inhibitory factor (LIF), c-kit ligand, thrombopoietin (TPO) and flt3 ligand. The present invention also includes culture conditions in which one or more cytokine is specifically excluded from the medium. Cytokines are commercially available from several vendors such as, for example, Genzyme (Framingham, Mass.), Genentech (South San Francisco, Calif.), Amgen (Thousand Oaks, Calif.), R&D Systems (Minneapolis, Minn.) and Immunex (Seattle, Wash.). It is intended, although not always explicitly stated, that molecules having similar biological activity as wild-type or purified cytokines (e.g., recombinantly produced or muteins thereof) are intended to be used within the spirit and scope of the invention.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

The term “peptide” or “polypeptide” are used interchangeably to refer to a compound of four or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics. A polypeptide of this invention may be as small as a minimal epitope for an antibody, e.g., having about 4 to about 8 amino acids or as long as a full length protein, alone or in combination with other proteins. In a preferred embodiment, one or more polypeptides of this invention will be combined, either as repeats of the same sequence or as a combination of different sequences.

The term “genetically modified” means containing and/or expressing a foreign gene or nucleic acid sequence which in turn, modifies the genotype or phenotype of the cell or its progeny. In other words, it refers to any addition, deletion or disruption to a cell's endogenous nucleotides.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989) supra). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

“Under transcriptional control” is a term well understood in the art and indicates that transcription of a polynucleotide sequence, usually a DNA sequence, depends on its being operably (operatively) linked to an element which contributes to the initiation of, or promotes, transcription. “Operably linked” refers to a juxtaposition wherein the elements are in an arrangement allowing them to function.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., WO 95/27071. Ads are easy to grow and do not require integration into the host cell genome. Recombinant Ad-derived vectors, particularly those that reduce the potential for recombination and generation of wild-type virus, have also been constructed. See, WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Zaks, et al. (1999) Nat. Med. 7:823-827.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in Current Protocols In Molecular Biology (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.gov/cgi-bin/BLAST.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

“In vitro” means outside the host subject's body and includes ex vivo.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

“Host cell,” “target cell” or “recipient cell” are intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, animal cells, and mammalian cells, e.g., murine, rat, simian or human.

A “subject” is a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”. For example, where the purpose of the experiment is to determine a correlation of an altered expression level of a gene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

“Suppressing” tumor growth indicates a growth state that is curtailed compared to growth without contact with educated, antigen-specific immune effector cells described herein. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and “suppressing” tumor growth indicates a growth state that is curtailed when stopping tumor growth, as well as tumor shrinkage.

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation and/or division of cells.

A “composition” is intended to mean a combination of an active agent and another compound or composition, inert (for example, a detectable agent or label) or active, such as an adjuvant. Additional amino acids combined with the active agent can be active or inert, and may include sequences to provide stability, targeting, enhanced immunogenicity, and the like.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15th Ed. (Mack Publ. Co., Easton (1975)).

An “effective amount” is an amount sufficient to effect beneficial or desired results. An effective amount can be administered in one or more administrations, applications or dosages.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein. Throughout this specification, numbering of amino acids in a peptide or polypeptide is from amino terminus to carboxy terminus.

The term “sequence motif” refers to a pattern present in a group of molecules. For instance, in one embodiment, the present invention provides for identification of a sequence motif among peptides. In this embodiment, a typical pattern may be identified by characteristic amino acid residues, such as hydrophobic, hydrophilic, basic, acidic, and the like.

“Heteroclitic peptide ligands” are defined in terms of function, i.e., they are more potent stimulators of the antigen-specific T cell clone than the parental antigen. T cell tolerance to an immunodominant T cell epitope can be overcome by immunization with heteroclitic cross-reactive peptide analogs of the tolerizing antigen.

Each lymphocyte carries cell surface antigen receptors of a single specificity, generated by random recombination of variable receptor gene segments and the pairing of different variable chains. This process ensures that the millions of lymphocytes in the body collectively carry millions of different antigen receptor specificities. This constitutes the antigen receptor repertoire of the individual.

During the lifetime of the individual only those lymphocytes that encounter an antigen to which it has specificity (i.e., bind) will be “activated” to proliferate and divide and give rise to a clone of identical progeny, all of whose receptors bind the same antigen. Antigen specificity is thus maintained as the progeny proliferate and differentiate into effector cells.

Because each lymphocyte has a different antigen-binding specificity, the fraction of lymphocytes that can bind and respond to any given antigen is very small. To generate sufficient specific effector lymphocytes to control a disease/infection, an activated lymphocyte must proliferate before its progeny finally differentiate into effector cells (i.e., clonal expansion).

Diversity within the T lymphocyte repertoire can be estimated by analyzing the distribution of T cell receptor (TCR) rearrangements.

Specific T cell recognition of MHC:peptide complexes is mediated via the TCR, a membrane-bound heterodimer composed of unique α- and β-chains with variable (V), diverse (D; β-chains only), joining (I), and constant (C) regions. Several factors contribute to TCR repertoire diversity, such as, the multiple possible V(D)J combinations generated during TCR gene rearrangement, the random mutations or nucleotide additions introduced at V(D)J junctions, and the random pairings of separately rearranged α- and β-chains. Individual V gene choice and the structure of the complementarity determining region 3 (CDR3) encoded by V(D)J junction sequences are thought to be critical determinants of TCR specificity.

This invention provide a method to select altered peptide species for administration to a subject presenting a native ligand, e.g., a tumor or viral antigen for the purpose of activating an immune response against the native ligand. Various methods are known to manufacture and isolate altered peptide species and the present inventions are not limited to any one method. The altered peptide species are designed and selected to active an immune response (T cell or B cell) against a native or cognate ligand. Many native ligands will not active an immune response due to self-tolerance or peripheral tolerance. In one aspect, the present method provides a means and compositions to break tolerance.

More than one or a plurality of altered peptide species are manufacture and screened for the ability to active an immune response. The screen can be an in vitro screen, examples of which are described herein, or may comprise an in vivo screen. Cell samples for use in the in vitro screen can be taken directly from a subject or can be cultured from a subject or commercially available. The population of altered peptides are then further assayed for the ability to activate populations of T cells, wherein at least two members of the population raise T cells with distinct T cell receptor Vβ recombinations. The screens encompass in vitro and in vivo assays. Methods to determine the Vβ sequence of a T cell or clones thereof are known in the art. See McMahan, C. J. and Fink, P. J. (2000) J. of Immun. 165:6902-6907; Kusaka, S. et al. (2000) J. of Immun. 164:2240-2247; and kalergis, A. M., et al. (1999) J. of Immun. 162:7263-7270. Cell samples for use in the in vitro screen can be taken directly from a subject or can be cultured from a subject or commercially available.

T cell receptor domains and B cell receptor domains share common ancestry. T cell receptors are comprised of an acidic alpha (α) chain and a basic beta (β) chain. The T cell receptor contains extracellular, transmembrane and cytoplasmic domains. The β chain contains variable (Vβ) and constant (Cβ) regions. The alpha and beta chains are different and are coded for by different genes. Rearrangement in the genes coding for the T cell receptor are characteristic of each T cell clone.

In one aspect, the altered peptides are selected based on the ability of each to activate different T cell clones from each other. In a further aspect, the altered peptides are selected based on the ability to activate a different subpopulation of CTLs. As above, this invention encompasses in vitro and in vivo screening methods for this selection step.

In a further aspect, the peptides are based on the ability to activate an immune response and to activate distinct T cell Vβ recombinations, T cell clones or CTLs in a single subject or in a population of subjects. Alternatively, the altered peptides are selected based on the ability to activate T cell populations having distinct T cell receptor Vβ recombination in a majority of the subjects comprising a population. In aspect, the subjects have a given HLA-type such as HLA-A2.

Various combinations of peptides can be selected. For example, at least two altered peptides, or at least three, or at least 2 to 6 altered peptides are selected.

The altered peptides are selected and can be combined in a carrier, e.g., a pharmaceutically acceptable carrier for administration to a subject. Alternatively, the peptides are present in a host cell. The host cell can be combined with a carrier, e.g., a pharmaceutically acceptable carrier.

Polynucleotides encoding the peptides are further provided, alone or in combination with a carrier, e.g., a pharmaceutically acceptable carrier. Vectors and host cells containing the polynucleotides are yet further provided by this invention. As above, the vectors and host cells can be combined with a carrier such as a pharmaceutically acceptable carrier.

The compositions of this invention are useful to modulate an immune response in a subject. In another aspect, they are useful to educate naïve immune effector cells. The combination of immune effector cell populations are futher provided by this invention. In one aspect, they are combined with a carrier, e.g.,a pharmaceutically acceptable carrier.

The compositions of this invention can modulate or alternatively activate or induce an immune response against a native ligand in a subject. Therefore, this invention also provides administration of the compositions to a subject to activate or induce an immune response against the native ligand.

In one aspect, this invention provides a method to provoke an immune response in a subject presenting a native ligand. The method requires activating a first population of immune effector cells educated by a first altered peptide in the subject, and then activating a second population of immune effector cells educated by a different altered peptide than the first altered peptide in the subject, whereby each of the activated immune effector cell populations: (i) elicits an immune response against the native antigen epitope; and (ii) have different and distinct T cell receptor Vβ recombinations in the subject. The method provides a means to provoke an immune response against the native ligand using a first population of immune effector cells and a second population of immune effector cells, each population specifically reacting with the native antigen yet distinct from each other in that the T cell receptor Vβ recombination of the first effector cell population is different from the T cell receptor Vβ recombination of the second effector cell population.

In one aspect, the method requires delivering to the subject an effective amount of a first and second altered ligand or “convergent altered ligand” (as defined herein) to the subject. The ligand can be delivered as a peptide or as a polynucleotide encoding the peptide. Polynucleotides can be delivered as described herein. The ligands and/or polypeptides can be delivered in a host cell, e.g., an antigen presenting cell such as a dendritic cell (DC). In a further aspect, an effective amount of a cytokine and/or co-stimulatory molecule is administered to the subject.

Alternatively, activation can be achieved by delivering an effective amount of a first and a second immune effector cell which have been educated in the presence and at the expense of a first and a second altered peptide, respectively.

Native ligands can be, but are not limited to, an antigenic epitope, a human tumor antigenic epitope, and a viral antigen epitope. This invention also provides a method for selecting a therapy for a subject such as a human patient. The method allows for the selection of altered ligands that specifically recognizes the native ligand in the subject.

The selected peptide species are delivered to the subject, wherein the delivery of said selected peptide species activates an immune response against said native ligand. In one aspect, each peptide species is administered individually. In another aspect, the selected peptide species are co-administered. In a further aspect, the selected altered ligands are capable of activating a heteroclitic immune response against said native ligand in the subject as compared with the administration of native peptide species or the administration of fewer altered peptide species.

In one aspect, the method requires delivering to the subject an effective amount of a first and second altered ligand or “convergent altered ligand (as defined herein) to the subject. The ligand can be delivered as a peptide or as a polynucleotide encoding the peptide. Polynucleotides can be delivered as described herein. In a further aspect, an effective amount of a cytokine and/or co-stimulatory molecule is administered to the subject. In yet a further aspect, the peptide and/or polynucleotide can be delivered in a host cell which in turn, is administered to the subject.

This invention also provides a composition containing selected multiple functionally convergent heteroclitic peptides directed at a single cognate native ligand, wherein the selection includes functionally convergent heteroclitic peptide ligands which collectively stimulate a T cell repertoire having plurality of T cell receptor Vβ recombinations. In other words, the composition of this invention contains at least two, isolated altered ligand species, wherein each of said species is characterized by an ability to elicit an immune response against the same cognate native ligand; and activate a different subpopulation of cytotoxic T lymphocytes (CTLs) against said native ligand. In other words, each species activates a different clonal population of CTLs. The isolated ligand species are preselected to provoke the mixed Vβ usage T cell repertoire in the subject while retaining the ability to selectively bind the native ligand. In a further aspect, from 2 to 100 altered peptide species is contained in the composition. Alternatively, at least 3 different altered peptide ligand species are contained in the composition. Ligand species include, but are not limited to tumor antigens, viral antigens or self-antigens.

In one embodiment, the composition includes an acceptable carrier or diluent and the peptide can be delivered as contiguous amino acids or alternatively, as polynucleotides encoding the peptides. In another embodiment, the altered peptide ligand is covalently linked to one or more amino acids naturally contiguous to said native human ligand.

Also provided by this invention is a kit comprising altered peptides or altered peptide ligands directed at a single cognate native ligand wherein a first altered ligand species activates a first T cell, a second altered ligand species activates a second T cell, and the T cell receptor Vβ recombination of said first T cell is different from the T cell receptor Vβ recombination of said second T cell; and instructions for the co-administration of each of said altered ligands to a subject, wherein said ligands are packaged alone or in combination, and wherein each of said ligands are characterized by an ability to elicit a different T cell receptor Vβ repertoire in a vertebrate subject. The kit contains peptides or polynucleotides encoding the peptides, and/or the polynucleotide and/or amino acid sequences for production of the peptides. In one aspect, the instructions further provide for the determination of the T cell receptor Vβ repertoire elicited by said subject. In a further aspect, the determination is conducted prior to and/or after said co-administration.

The following examples are intended to illustrate, and not limit the invention.

Methods for Designing Altered Peptide Ligands

Altered peptide ligands can be designed based on natural peptide epitopes identified using any method known in the art. The following provides non-limiting examples of such methods. In addition, modifications or combinations of any of the following methods can be used.

Methods involving isolating and assaying MHC molecules from antigen presenting cells can be used to identify peptides bound to the MHC molecules. Chicz and Urban (1994) Immunol. Today 1-5:155-160. Bacteriophage “phage display” libraries can also be constructed. Using the “phage method” (Scott and Smith (1990) Science 249:386-390; Cwirla et al. (1990) Proc. Natl. Acad. Sci. USA 87:6378-6382; Devlin et al. (1990) Science 249:404-406), very large libraries can be constructed (10⁶-10⁸ chemical entities). Other methods to identify peptide epitopes which can be used involve primarily chemical methods, of which the Geysen method (Geysen et al. (1986) Molecular Immunology 23:709-715; Geysen et al. (1987) J. Immunologic Method 102:259-274; and the method of Fodor et al. (1991) Science 251:767-773) are examples. Furka et al. (1988) 14th International Congress of Biochemistry, Volume 5. Abstract FR:013; Furka, (1991) Int. J. Peptide Protein Res. 37:487-493). Houghton (U.S. Pat. No. 4,631,211 issued December 1986) and Rutter et al. (U.S. Pat. No. 5,101,175, issued Apr. 23, 1991) describe methods to produce a mixture of peptides that can be tested as agonists or antagonists. Other methods which can be employed involve use of synthetic libraries (Needels et al. (1993) Proc. Natl. Acad. Sci. USA 90:10700-4; Ohlmeyer et al. (1993) Proc. Natl. Acad. Sci. USA 90:10922-10926; Lam et al., International Patent Publication No. WO 92/00252, each of which is incorporated herein by reference in its entirety), and the like can be used to screen for receptor ligands. Techniques based on cDNA subtraction or differential display have been described amply in the literature and can also be used. see, for example, Hedrick et al. (1984) Nature 308:149; and Lian and Pardee (1992) Science 257:967. The expressed sequence tag (EST) approach is a valuable tool for gene discovery (Adams et al. (1991) Science 252:1651), as are Northern blotting, RNase protection, and reverse transcriptase-polymerase chain reaction (RT-PCR) analysis (Alwine et al. (1977) Proc. Natl. Acad. Sci. USA 74:5350; Zinn et al. (1983) Cell 34:865; Veres et al. (1987) Science 237:415). Another technique which can be used is the “pepscan” technique (Van der Zee (1989) Eur. J. Immunol. 19:43-47) in which several dozens of peptides are simultaneously synthesized on polyethylene rods arrayed in a 96-well microtiter plate pattern, similar to an indexed library in that the position of each pin defines the synthesis history on it. Peptides are then chemically cleaved from the solid support and supplied to irradiated syngeneic thymocytes for antigen presentation. A cloned CTL line is then tested for reactivity in a proliferation assay monitored by ³H-thymidine incorporation.

SPHERE is described in WO 97/35035. This approach utilizes combinatorial peptide libraries synthesized on polystyrene beads wherein each bead contains a pure population of a unique peptide that can be chemically released from the beads in discrete aliquots. Released peptide from pooled bead arrays are screened using methods to detect T cell activation, including, for example, ³H-thymidine incorporation (for CD4⁺ or CD8⁺ T cells), ⁵¹Cr-release assay (for CTLs) or IL-2 production (for CD4⁺ T cells) to identify peptide pools capable of activating a T cell of interest. By utilizing an iterative peptide pool/releasing strategy, it is possible to screen more than 10⁷ peptides in just a few days. Analysis of residual peptide on the corresponding positive beads (>100 pmoles) allows rapid and unambiguous identification of the epitope sequence.

A brief overview of an assay to identify peptides binding to CTLs is as follows: roughly speaking, ten 96-well plates with 1000 beads per well will accommodate 10⁶ beads; ten 96-well plates with 100 beads per well will accommodate 10⁵ beads. In order to minimize both the number of CTL cells required per screen and the amount of manual manipulations, the eluted peptides can be further pooled to yield wells with any desired complexity. For example, based on experiments with soluble libraries, it is possible to screen 10⁷ peptides in 96-well plates (10,000 peptides per well) with as few as 2×10⁶ CTL cells. After cleaving a percentage of the peptides from the beads, incubating them with gamma-irradiated foster APCs and the cloned CTL line(s), positive wells determined by ³H-thymidine incorporation are further examined. Alternatively, as pointed out above, cytokine production or cytolytic ⁵¹ Cr-release assays maybe used. Coulie et al. (1992) Int. J. Cancer 50:289-291. Beads from each positive well will be separated and assayed individually as before, utilizing an additional percentage of the peptide from each bead. Positive individual beads will then be decoded, identifying the reactive amino acid sequence. Analysis of all positives will give a partial profile of conservatively substituted epitopes that stimulate the CTL clone tested. At this point, the peptide can be resynthesized and retested. Also, a second library (of minimal complexity) can be synthesized with representations of all conservative substitutions in order to enumerate the complete spectrum of derivatives tolerated by a particular CTL. By screening multiple CTLs (of the same MHC restriction) simultaneously, the search for crossreacting epitopes is greatly facilitated.

The described method for the identification of CD8⁺ MHC Class I-restricted CTL epitopes can be applied to the identification of CD4⁺ MHC Class II-restricted CD4⁺ T cell epitopes. In this case, MHC Class II allele-specific libraries are synthesized such that haplotype-specific anchor residues are represented at the appropriate positions. MHC Class II agretopic motifs have been identified for the common alleles. Rammensee (1995) Curr. Opin. Immunol. 7:85-96; Altuvia et al. (1994) Mol. Immunol. 24:375-379; Reay et al. (1994) J. Immunol. 152:3946-3957; Verreck et al. (1994) Eur. J. Immunol. 24:375-379; Sinigaglia and Hammer (1994) Curr. Opin. Immunol. 6:52-56; Rotzschke and Falk (1994) Curr. Opin. Immunol. 6:45-51. The overall length of the peptides will be 12-20 amino acid residues, and previously described methods may be employed to limit library complexity.

Production of Altered Peptide Ligands

The peptides used in accordance with the method of the present invention can be obtained in any one of a number of conventional ways. Because they will generally be short sequences, they can be prepared by chemical synthesis using standard techniques. Particularly convenient are solid phase peptide synthesis techniques. Automated peptide synthesizers are commercially available, as are the reagents required for their use. Alternatively, the peptides can be prepared by enzymatic digestion or cleavage of naturally occurring proteins. The peptides can also be prepared using recombinant techniques known to those of skill in the art.

In one embodiment, isolated altered peptide ligands of the present invention can be synthesized using an appropriate solid state synthetic procedure. Steward and Young, eds. (1968) “Solid Phase Peptide Synthesis” Freemantle, San Francisco, Calif. A preferred method is the Merrifield process. Merrifield (1967) Recent progress in Hormone Res. 23:451. The antigenic activity of these peptides may conveniently be tested using, for example, the assays described herein.

Once an isolated peptide of the invention is obtained, it may be purified by standard methods including chromatography (e.g., ion exchange, affinity, and sizing column chromatography), centrifugation, differential solubility, or by any other standard technique for protein purification. For immunoaffinity chromatography, an epitope may be isolated by binding it to an affinity column comprising antibodies that were raised against that peptide, or a related peptide of the invention, and were affixed to a stationary support.

Alternatively, affinity tags such as hexa-His (Invitrogen), Maltose binding domain (New England Biolabs), influenza coat sequence (Kolodziej et al. (1991) Methods Enzymol. 194:508-509), and glutathione-S-transferase can be attached to the peptides of the invention to allow easy purification by passage over an appropriate affinity column. Isolated peptides can also be physically characterized using such techniques as proteolysis, nuclear magnetic resonance, and x-ray crystallography.

Peptide Formulations

The altered peptide ligands of the invention can be used in a variety of formulations, which may vary depending on the intended use. They can be covalently or non-covalently linked (complexed) to various other molecules, the nature of which may vary depending on the particular purpose. For example, altered peptide ligands of the invention can be covalently or non-covalently complexed to a macromolecular carrier, including, but not limited to, natural and synthetic polymers, proteins, polysaccharides, poly(amino acid), polyvinyl alcohol, polyvinyl pyrrolidone, and lipids. A peptide can be conjugated to a fatty acid, for introduction into a liposome. U.S. Pat. No. 5,837,249. Altered peptide ligands can be complexed covalently or non-covalently with a solid support, a variety of which are known in the art. Altered peptide ligands also can be associated with an antigen-presenting matrix with or without co-stimulatory molecules, as described in more detail below.

Examples of protein carriers include, but are not limited to, superantigens, serum albumin, tetanus toxoid, ovalbumin, thyroglobulin, myoglobulin, and immunoglobulin.

Peptide-protein carrier polymers may be formed using conventional crosslinking agents such as carbodiimides. Examples of carbodiimides are 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-44-dimethylpentyl) carbodiimide.

Examples of other suitable crosslinking agents are cyanogen bromide, glutaraldehyde and succinic anhydride. In general, any of a number of homobifunctional agents including a homobifunctional aldehyde, a homobifunctional epoxide, a homobifunctional imidoester, a homobifunctional N-hydroxysuccinimide ester, a homobifunctional maleimide, a homobifunctional alkyl halide, a homobifunctional pyridyl disulfide, a homobifunctional aryl halide, a homobifunctional hydrazide, a homobifunctional diazonium derivative and a homobifunctional photoreactive compound may be used. Also included are heterobifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homobifunctional crosslinking agents include the bifunctional N-hydroxysuccinimide esters dithiobis(succinimidylpropionate), disuccinimidyl suberate, and disuccinimidyl tartarate; the bifunctional imidoesters dimethyl adipimidate, dimethyl pimelimidate, and dimethyl suberimidate; the bifunctional sulfhydryl-reactive crosslinkers 1,4-di-[3 ′-(2′-pyridyldithio) propion-amido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether, the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri(2-chloroethyl)amine, respectively.

Examples of other common heterobifunctional cross-linking agents that may be used to effect the conjugation of proteins to peptides include, but are not limited to, SMCC succinimidyl-4-(N-maleimidomethyl)cyclohexane-1-carboxylate), MBS (m-maleimidobenzoyl-N-hydroxysuccinimide ester), SLAB (N-succinimidyl(4-iodoacteyl)aminobenzoate), SMPB (succinimidyl-4-(p-maleimidophenyl)butyrate), GMBS (N-(γ-maleimidobutyryloxy)succinimide ester), MPBH (4-(4-N-maleimidopohenyl) butyric acid hydrazide), M2C2H (4-(N-maleimidomethyl) cyclohexane-1-carboxyl-hydrazide), SMPT (succinimidyloxycarbonyl-α-methyl-α-(2-pyridyldithio)toluene), and SPDP (N-succinimidyl 3-(2-pyridyldithio)propionate).

Crosslinking may be accomplished by coupling a carbonyl group to an amine group or to a hydrazide group by reductive amination.

Altered peptide ligands also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to, Freund's Complete and Incomplete, mineral salts and polynucleotides.

Polynucleotides Comprising Sequences Encoding Altered Peptide Ligands

The invention further provides isolated polynucleotides encoding altered peptide ligands. As used herein, the term “polynucleotide” encompasses DNA, RNA and nucleic acid mimetics. In addition to the polynucleotide sequences encoding a ligand of the invention, or their complements, this invention also provides the anti-sense polynucleotide strand, e.g., antisense RNA to these sequences or their complements. One can obtain an antisense RNA using known sequences and the methodology described in Vander Krol et al. (1988) BioTechniques 6:958.

The polynucleotides can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmental undesirable reagents. In the case of enzyme tags, colorimetric indicator substrates are known which can be employed to provide a means visible to the human eye or spectrophotometrically, to identify specific hybridization with complementary nucleic acid-containing samples. Briefly, this invention further provides a method for detecting a single-stranded polynucleotide or its complement, by contacting target single-stranded polynucleotides with a labeled, single-stranded polynucleotide (a probe) which is at least 4, and more preferably at least 5 or 6 and most preferably at least 10 of the 10 nucleotides of a polynucleotide of the invention (or the corresponding complement) under conditions permitting hybridization (preferably moderately stringent hybridization conditions) of complementary single-stranded polynucleotides, or more preferably, under highly stringent hybridization conditions. Hybridized polynucleotide pairs are separated from un-hybridized, single-stranded polynucleotides. The hybridized polynucleotide pairs are detected using methods well known to those of skill in the art and set forth, for example, in Sambrook et al. (1989) supra.

The polynucleotides of this invention can be replicated using PCR. PCR technology is the subject matter of U.S. Pat. Nos. 4,683,195, 4,800,159, 4,754,065, and 4,683,202 and described in PCR: THE POLYMERASE CHAIN REACTION (Mullis et al. eds., Birkhauser Press, Boston (1994)) and references cited therein.

Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

RNA can be obtained by first inserting a DNA polynucleotide into a suitable host cell. The DNA can be inserted by any appropriate method, e.g., by the use of an appropriate gene delivery vehicle (e.g. liposome, plasmid or vector) or by electroporation. When the cell replicates and the DNA is transcribed into RNA; the RNA can then be isolated using methods well known to those of skill in the art, for example, as set forth in Sambrook et al. (1989) supra. For instance, mRNA can be isolated using various lytic enzymes or chemical solutions according to the procedures set forth in Sambrook et al. (1989) supra or extracted by nucleic-acid-binding resins following the accompanying instructions provided by manufactures.

It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. Preferably, a probe useful for detecting the aforementioned mRNA is at least about 80% identical to the homologous region of comparable size. More preferably, the probe is 85% identical to the corresponding gene sequence after alignment of the homologous region; even more preferably, it exhibits 90% identity.

These probes can be used in radioassays (e.g., Southern and Northern blot analysis) to detect or monitor various cells or tissue containing these cells. The probes also can be attached to a solid support or an array such as a chip for use in high throughput screening assays for the detection of expression of the gene corresponding to one or more polynucleotide(s) of this invention.

The invention further provides the isolated polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are well known in the art and commercially available. For general methodology and cloning strategies, see “Gene Expression Technology” (Goeddel ed., Academic Press, Inc. (1991)) and references cited therein and “Vectors: Essential Data Series” (Gacesa and Ramji, eds., John Wiley & Sons, NY (1994)), which contains maps, functional properties, commercial suppliers and a reference to GenEMBL accession numbers for various suitable vectors. Preferably, these vectors are capable of transcribing RNA in vitro or in vivo.

Delivery Vehicles Comprising A Polynucleotide Encoding the Altered Peptide Ligand

The present invention also provides delivery vehicles suitable for delivery of a polynucleotide of the invention into cells (whether in vivo, ex vivo, or in vitro). A polynucleotide of the invention can be contained within a cloning or expression vector. These vectors (especially expression vectors) can in turn be manipulated to assume any of a number of forms that may, for example, facilitate delivery to and/or entry into a cell.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using well known methods. See Sambrook, et al. (1989) supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; or DEAE-dextran; electroporation; or microinjection. See Sambrook et al. (1989) supra for this methodology. Thus, this invention also provides a host cell, e.g., a mammalian cell, an animal cell (rat or mouse), a human cell, or a prokaryotic cell such as a bacterial cell, containing a polynucleotide encoding a protein or polypeptide or antibody.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6. Miller et al. (1989) BioTechniques 7:980-990. The methodology of using replication-incompetent retroviruses for retroviral-mediated gene transfer of gene markers is well established. Correll et al. (1989) Proc. Natl. Acad. Sci. USA 86:8912; Bordignon (1989) Proc. Natl. Acad. Sci. USA 86:8912-52; Culver (1991) Proc. Natl. Acad. Sci. USA 88:3155; and Rill (1991) Blood 79(10):2694-700.

In general, genetic modifications of cells employed in the present invention are accomplished by introducing a vector containing a polynucleotide comprising sequences encoding one or more altered peptide ligand(s) of the invention. A variety of different gene transfer vectors, including viral as well as non-viral systems can be used.

A wide variety of non-viral vehicles for delivery of a polynucleotide of the invention are known in the art and are encompassed in the present invention. A polynucleotide of the invention can be delivered to a cell as naked DNA. WO 97/40163. Alternatively, a polynucleotide of the invention can be delivered to a cell associated in a variety of ways with a variety of substances (forms of delivery) including, but not limited to cationic lipids; biocompatible polymers, including natural polymers and synthetic polymers; lipoproteins; polypeptides; polysaccharides; lipopolysaccharides; artificial viral envelopes; metal particles; and bacteria. A delivery vehicle may take the form of a microparticle. Mixtures or conjugates of these various substances can also be used as delivery vehicles. A polynucleotide of the invention can be associated with these various forms of delivery non-covalently or covalently.

Included in the non-viral vector category are prokaryotic plasmids and eukaryotic plasmids. Non-viral vectors (i.e., cloning and expression vectors) having cloned therein a polynucleotide(s) of the invention can be used for expression of recombinant polypeptides as well as a source of polynucleotide of the invention. Cloning vectors can be used to obtain replicate copies of the polynucleotides they contain, or as a means of storing the polynucleotides in a depository for future recovery. Expression vectors (and host cells containing these expression vectors) can be used to obtain polypeptides produced from the polynucleotides they contain. They may also be used where it is desirable to express polypeptides, encoded by an operably linked polynucleotide, in an individual, such as for eliciting an immune response via the polypeptide(s) encoded in the expression vector(s). Suitable cloning and expression vectors include any known in the art, e.g., those for use in bacterial, mammalian, yeast and insect expression systems. Specific vectors and suitable host cells are known in the art and need not be described in detail herein. For example, see Gacesa and Ramji, Vectors, John Wiley & Sons (1994).

Cloning and expression vectors typically contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins substances, e.g. ampicillin, neomycin, methotrexate, etc.; (b) complement auxotrophic deficiencies; or (c) supply critical nutrients not available from complex media. The choice of the proper marker gene will depend on the host cell, and appropriate genes for different hosts are known in the art. Cloning and expression vectors also typically contain a replication system recognized by the host.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Stratagene, and Invitrogen.

Expression vectors generally are replicable polynucleotide constructs that contain a polynucleotide encoding a polypeptide of interest. The polynucleotide encoding the polypeptide of interest is operably linked to suitable transcriptional controlling elements, such as promoters, enhancers and terminators. For expression (i.e., translation), one or more translational controlling elements are also usually required, such as ribosome binding sites, translation initiation sites, and stop codons. A polynucleotide sequence encoding a signal peptide can also be included to allow a polypeptide, encoded by an operably linked polynucleotide, to cross and/or lodge in cell membranes or be secreted from the cell. A number of expression vectors suitable for expression in eukaryotic cells including yeast, avian, and mammalian cells are known in the art. Examples of mammalian expression vectors contain both prokaryotic sequence to facilitate the propagation of the vector in bacteria, and one or more eukaryotic transcription units that are expressed in eukaryotic cells. Examples of mammalian expression vectors suitable for transfection of eukaryotic cells include the pcDNAI/amp, pcDNAI/neo, pRc/CMV, pSV2gpt, pSV2neo, pRSVneo, and pHyg derived vectors. Alternatively, derivatives of viruses such as the bovine papilloma virus (BPV-1), or Epstein-Barr virus (pHEB, pREP derived vectors) can be used for expression in mammalian cells. Examples of expression vectors for yeast systems, include YEP24, YIP5, YEP51, YEP52, YES2 and YRP17, which are cloning and expression vehicles useful for introduction of constructs into S. cerevisiae. Broach et al. (1983) “Experimental Manipulation of Gene Expression” ed. M. Inouye, Academic Press. p. 83. Baculovirus expression vectors for expression in insect cells include pVL-derived vectors (such as pVL1392, pVL1393 and pVL941), pAcUW-derived vectors and pBlueBac-derived vectors.

Viral vectors include, but are not limited to, DNA viral vectors such as those based on adenoviruses, herpes simplex virus, poxviruses such as vaccinia virus, and parvoviruses, including adeno-associated virus; and RNA viral vectors, including, but not limited to, the retroviral vectors. Retroviral vectors include murine leukemia virus, and lentiviruses such as human immunodeficiency virus. Naldini et al. (1996) Science 272:263-267.

Replication-defective retroviral vectors harboring a polynucleotide of the invention as part of the retroviral genome can be used. Such vectors have been described in detail. (Miller et al. (1990) Mol. Cell Biol. 10:4239; Kolberg, R. (1992) J. NIH Res. 4:43; Cornetta et al. (1991) Hum. Gene Therapy 2:215).

Adenovirus and adeno-associated virus vectors useful in the genetic modifications of this invention may be produced according to methods already taught in the art. (See, e.g., Karlsson et al. (1986) EMBO 5:2377; Carter (1992) Current Opinion in Biotechnology 3:533-539; Muzcyzka (1992) Current Top. Microbiol. Immunol. 158:97-129; “Gene Targeting: A Practical Approach” (1992) ed. A. L. Joyner, Oxford University Press, NY). Several different approaches are feasible.

Additional references describing viral vectors which could be used in the methods of the present invention include the following: Horwitz, M. S., Adenoviridae and Their Replication, in Fields, B., et al. (eds.) “Virology”, Vol. 2, Raven Press New York, pp. 1679-1721, 1990); Graham, F. et al., pp. 109-128 in “Methods In Molecular Biology”, Vol. 7: “Gene Transfer And Expression Protocols”, Murray, E. (ed.), Humana Press, Clifton, N. J. (1991); Miller et al. (1995) FASEB Journal 9:190-199, Schreier (1994) Pharmaceutica Acta Helvetiae 68:145-159; Schneider and French (1993) Circulation 88:1937-1942; Curiel et al. (1992) Human Gene Therapy 3:147-154; Graham et al. WO 95/00655 (Jan. 5, 1995); Falck-Pedersen WO 95/16772 (Jun. 22, 1995); Denefle et al. WO 95/23867 (Sep. 8, 1995); Haddada et al. WO 94/26914 (Nov. 24, 1994); Perricaudet et al. WO 95/02697 (Jan. 26, 1995); and Zhang et al. WO 95/25071 (Oct. 12, 1995).

The efficiency of transduction of DCs or other APCs can be assessed by immunofluorescence using fluorescent antibodies specific for the tumor antigen being expressed (Kim et al. (1997) J. Immunother. 20:276-286). Alternatively, the antibodies can be conjugated to an enzyme (e.g., HRP) giving rise to a colored product upon reaction with the substrate. The actual amount of antigenic polypeptides being expressed by the APCs can be evaluated by ELISA.

In vivo transduction of DCs, or other APCs, can be accomplished by administration of a viral vector comprising a polynucleotide of the invention via different routes including intravenous, intramuscular, intranasal, intraperitoneal or cutaneous delivery. One method which can be used is cutaneous delivery of Ad vector at multiple sites using a total dose of approximately 1×10¹⁰−1×10¹² i.u. Levels of in vivo transduction can be roughly assessed by co-staining with antibodies directed against APC marker(s) and the peptide epitope being expressed. The staining procedure can be carried out on biopsy samples from the site of administration or on cells from draining lymph nodes or other organs where APCs (in particular DCs) may have migrated. Condon et al. (1996) Nature Med. 2:1122-1128; Wan et al. (1997) Human Gene Therapy 8:1355-1363. The amount of antigen being expressed at the site of injection or in other organs where transduced APCs may have migrated can be evaluated by ELISA on tissue homogenates.

APCs can also be transduced in vitro/ex vivo by non-viral gene delivery methods such as electroporation, calcium phosphate precipitation or cationic lipid/plasmid DNA complexes. Arthur et al. (1997) Cancer Gene Therapy 4:17-25. Transduced APCs can subsequently be administered to the host via an intravenous, subcutaneous, intranasal, intramuscular or intraperitoneal route of delivery.

In vivo transduction of DCs, or other APCs, can potentially be accomplished by administration of cationic lipid/plasmid DNA complexes delivered via the intravenous, intramuscular, intranasal, intraperitoneal or cutaneous route of administration. Gene gun delivery or injection of naked plasmid DNA into the skin also leads to transduction of DCs. Condon et al. (1996) Nature Med. 2:1122-1128; Raz et al. (1994) Proc. Natl. Acad. Sci. USA 91:9519-9523. Intramuscular delivery of plasmid DNA may also be used for immunization. Rosato et al. (1997) Human Gene Therapy 8:1451-1458.

The transduction efficiency and levels of transgene expression can be assessed as described above for viral vectors.

Host Cells Comprising Polynucleotides Encoding Altered Peptide Ligands

The present invention further provides host cells comprising polynucleotides of the invention. Host cells containing the polynucleotides of this invention are useful for the recombinant replication of the polynucleotides and for the recombinant production of peptides of the invention. Alternatively, host cells comprising a polynucleotide of the invention may be used to induce an immune response in a subject in the methods described herein.

Host cells which are suitable for recombinant replication of the polynucleotides of the invention, and for the recombinant production of peptides of the invention can be prokaryotic or eukaryotic. Host systems are known in the art and need not be described in detail herein. Prokaryotic hosts include bacterial cells, for example E. coli, B. subtilis, and mycobacteria. Among eukaryotic hosts are yeast, insect, avian, plant, C. elegans (or nematode) and mammalian cells. These cells are cultured in conventional nutrient media modified as appropriate for inducing promoters, selecting transformants, or amplifying the genes encoding the desired sequences.

When the host cells are antigen presenting cells, they can be used to expand a population of immune effector cells such as tumor infiltrating lymphocytes which in turn are useful in adoptive immunotherapies. Antigen presenting cells are described in more detail below.

Host Cells Presenting Altered Peptide Ligands

The invention further provides isolated host cells comprising altered peptide ligands. In some embodiments, these host cells present two or more peptides of the invention on the surface of the cell in the context of an MHC molecule such that the peptide can be recognized by an immune effector cell. Isolated host cells which present the polypeptides of this invention in the context of MHC molecules are further useful to expand and isolate a population of educated, antigen-specific immune effector cells. The immune effector cells, e.g., cytotoxic T lymphocytes, are produced by culturing naive immune effector cells with antigen-presenting cells which present the polypeptides in the context of MHC molecules on the surface of the APCs. The population can be purified using methods known in the art, e.g., FACS analysis or FICOLL™ gradient. The methods to generate and culture the immune effector cells as well as the populations produced thereby also are the inventor's contribution and invention. Pharmaceutical compositions comprising the cells and pharmaceutically acceptable carriers are useful in adoptive immunotherapy. Prior to administration in vivo, the immune effector cells are screened in vitro for their ability to target cells.

In some of these embodiments, isolated host cells are APCs. APCs include, but are not limited to, dendritic cells (DCs), monocytes/macrophages, B lymphocytes or other cell type(s) expressing the necessary MHC/co-stimulatory molecules.

In some embodiments, the immune effector cells and/or the APCs are genetically modified. Using standard gene transfer, genes coding for co-stimulatory molecules and/or stimulatory cytokines can be inserted prior to, concurrent to or subsequent to expansion of the immune effector cells.

APCs can obtained from a variety of sources, including but not limited to, peripheral blood mononuclear cells (PBMC), whole blood or fractions thereof containing mixed populations, spleen cells, bone marrow cells, tumor infiltrating lymphocytes, cells obtained by leukapheresis, lymph nodes, e.g., lymph nodes draining from a tumor. Suitable donors include an immunized donor, a non-immunized (naïve) donor, treated or untreated donors. A “treated” donor is one that has been exposed to one or more biological modifiers. An “untreated” donor has not been exposed to one or more biological modifiers. APCs can also be treated in vitro with one or more biological modifiers.

The APCs are generally alive but can also be irradiated, mitomycin C treated, attenuated, or chemically fixed. Further, the APCs need not be whole cells. Instead, vesicle preparations of APCs can be used.

APCs can be genetically modified, i.e., transfected with a recombinant polynucleotide construct such that they express a polypeptide or an RNA molecule which they would not normally express or would normally express at lower levels. Examples of polynucleotides include, but are not limited to, those which encode an MHC molecule; a co-stimulatory molecule such as B7; and a peptide or polypeptide of the invention.

Cells which do not normally function in vivo in mammals as APCs can be modified in such a way that they function as APCs. A wide variety of cells can function as APCs when appropriately modified. Examples of such cells are insect cells, for example Drosophila or Spodoptera; and foster cells, such as the human cell line T2. For example, expression vectors which direct the synthesis of one or more antigen-presenting polypeptides, such as MHC molecules, optionally also accessory molecules such as B7, can be introduced into these cells to effect the expression on the surface of these cells antigen presentation molecules and, optionally, accessory molecules or functional portions thereof. Alternatively, antigen-presenting polypeptides and accessory molecules which can insert themselves into the cell membrane can be used. For example, glycosyl-phosphotidylinositol (GPI)-modified polypeptides can insert themselves into the membranes of cells. Hirose et al. (1995) Methods Enzymol. 250:582-614; and Huang et al. (1994) Immunity 1:607-613. Accessory molecules include, but are not limited to, co-stimulatory antibodies such as antibodies specific for CD28, CD80, or CD86; costimulatory molecules, including, but not limited to, B7.1 and B7.2; adhesion molecules such as ICAM-1 and LFA-3; and survival molecules such as Fas ligand and CD70. See, for example, PCT Publication No. WO 97/46256.

Foster antigen presenting cells are particularly useful as APCs. Foster APCs are derived from the human cell line 174×CEM.T2, referred to as T2, which contains a mutation in its antigen processing pathway that restricts the association of endogenous peptides with cell surface MHC class I molecules. Zweerink et al. (1993) J. Immunol. 150:1763-1771. This is due to a large homozygous deletion in the MHC class II region encompassing the genes TAP1, TAP2, LMP1, and LMP2, which are required for antigen presentation to MHC class 1-restricted CD8⁺ CTLs. In effect, only “empty” MHC class I molecules are presented on the surface of these cells. Exogenous peptide added to the culture medium binds to these MHC molecules provided that the peptide contains the allele-specific binding motif. These T2 cells are referred to herein as “foster” APCs. They can be used in conjunction with this invention to present antigen(s).

Transduction of T2 cells with specific recombinant MHC alleles allows for redirection of the MHC restriction profile. Libraries tailored to the recombinant allele will be preferentially presented by them because the anchor residues will prevent efficient binding to the endogenous allele.

High level expression of MHC molecules makes the APC more visible to the CTLs. Expressing the MHC allele of interest in T2 cells using a powerful transcriptional promoter (e.g., the CMV promoter) results in a more reactive APC (most likely due to a higher concentration of reactive MHC-peptide complexes on the cell surface).

The following is a brief description of two fundamental approaches for the isolation of APC. These approaches involve (1) isolating bone marrow precursor cells (CD34⁺) from blood and stimulating them to differentiate into APC; or (2) collecting the precommitted APCs from peripheral blood. In the first approach, the patient must be treated with cytokines such as GM-CSF to boost the number of circulating CD34⁺ stem cells in the peripheral blood.

The second approach for isolating APCs is to collect the relatively large numbers of precommitted APCs already circulating in the blood. Previous techniques for isolating committed APCs from human peripheral blood have involved combinations of physical procedures such as metrizamide gradients and adherence/nonadherence steps (Freudenthal et al. (1990) Proc. Natl. Acad. Sci. USA 87:7698-7702); Percoll gradient separations (Mehta-Damani et al. (1994) J. Immunol. 153:996-1003); and fluorescence activated cell sorting techniques (Thomas et al. (1993) J. Immunol. 151:6840-52).

One technique for separating large numbers of cells from one another is known as countercurrent centrifugal elutriation (CCE). In this technique, cells are subject to simultaneous centrifugation and a washout stream of buffer which is constantly increasing in flow rate. The constantly increasing countercurrent flow of buffer leads to fractional cell separations that are largely based on cell size.

In one aspect of the invention, the APC are precommitted or mature dendritic cells which can be isolated from the white blood cell fraction of a mammal, such as a murine, simian or a human (See, e.g., WO 96/23060). The white blood cell fraction can be from the peripheral blood of the mammal. This method includes the following steps: (a) providing a white blood cell fraction obtained from a mammalian source by methods known in the art such as leukapheresis; (b) separating the white blood cell fraction of step (a) into four or more subfractions by countercurrent centrifugal elutriation, (c) stimulating conversion of monocytes in one or more fractions from step (b) to dendritic cells by contacting the cells with calcium ionophore, GM-CSF and IL-13 or GM-CSF and IL-4, (d) identifying the dendritic cell-enriched fraction from step (c), and (e) collecting the enriched fraction of step (d), preferably at about 4° C. One way to identify the dendritic cell-enriched fraction is by fluorescence-activated cell sorting. The white blood cell fraction can be treated with calcium ionophore in the presence of other cytokines, such as recombinant (rh) rhIL-12, rhGM-CSF, or rhIL-4. The cells of the white blood cell fraction can be washed in buffer and suspended in Ca⁺⁺/Mg⁺⁺ free media prior to the separating step. The white blood cell fraction can be obtained by leukapheresis. The dendritic cells can be identified by the presence of at least one of the following markers: HLA-DR, HLA-DQ, or B7. 2, and the simultaneous absence of the following markers: CD3, CD14, CD16, 56, 57, and CD 19, 20. Monoclonal antibodies specific to these cell surface markers are commercially available.

More specifically, the method requires collecting an enriched collection of white cells and platelets from leukapheresis that is then further fractionated by countercurrent centrifugal elutriation (CCE). Abrahamsen et al. (1991) J. Clin. Apheresis 6:48-53. Cell samples are placed in a special elutriation rotor. The rotor is then spun at a constant speed of, for example, 3000 rpm. Once the rotor has reached the desired speed, pressurized air is used to control the flow rate of cells. Cells in the elutriator are subjected to simultaneous centrifugation and a washout stream of buffer which is constantly increasing in flow rate. This results in fractional cell separations based largely but not exclusively on differences in cell size.

Quality control of APC and more specifically DC collection and confirmation of their successful activation in culture is dependent upon a simultaneous multi-color FACS analysis technique which monitors both monocytes and the dendritic cell subpopulation as well as possible contaminant T lymphocytes. It is based upon the fact that DCs do not express the following markers: CD3 (T cell); CD14 (monocyte); CD16, 56, 57 (NK/LAK cells); CD19, 20 (B cells). At the same time, DCs do express large quantities of HLA-DR, significant HLA-DQ and B7.2 (but little or no B7.1) at the time they are circulating in the blood (in addition they express Leu M7 and M9, myeloid markers which are also expressed by monocytes and neutrophils).

Once collected, the DC rich/monocyte APC fractions (usually 150 through 190) can be pooled and cryopreserved for future use, or immediately placed in short term culture.

Alternatively, others have reported that a method for upregulating (activating) dendritic cells and converting monocytes to an activated dendritic cell phenotype. This method involves the addition of calcium ionophore to the culture media convert monocytes into activated dendritic cells. Adding the calcium ionophore A23187, for example, at the beginning of a 24-48 hour culture period resulted in uniform activation and dendritic cell phenotypic conversion of the pooled “monocyte plus DC” fractions: characteristically, the activated population becomes uniformly CD14 (Leu M3) negative, and upregulates HLA-DR, HLA-DQ, ICAM-1, B7.1, and B7.2.

Specific combination(s) of cytokines have been used successfully to amplify (or partially substitute) for the activation/conversion achieved with calcium ionophore: these cytokines include but are not limited to purified or recombinant human (“rh”) rhGM-CSF, rhIL-2, and rhIL-4. Each cytokine when given alone is inadequate for optimal upregulation.

Presentation of Altered Peptide Ligands by Antigen-Presenting Matrices

For use in immunomodulatory methods and diagnostic methods of the invention, an antigen-presenting matrix presents convergent antigenic peptide ligands of the invention bound to an MHC molecule. Any known method can be used to achieve presentation by an antigen-presenting matrix. The following are non-limiting examples of methods that can be used.

Altered peptide ligands can be delivered to antigen-presenting cells as polypeptide or peptide or in the form of cDNA encoding the protein/peptide.

Another method to deliver a synthetic antigenic peptide epitope of the invention to an APC is by pulsing. Pulsing can be accomplished in vitro/ex vivo by exposing APCs to the antigenic polypeptide(s) or peptide(s) of this invention. The polypeptide(s) or peptide(s) are added to APCs at a concentration of 1-10 μm for approximately 3 hours. Pulsed APCs can subsequently be administered to the host via an intravenous, subcutaneous, intranasal, intramuscular or intraperitoneal route of delivery.

Altered peptide ligands can also be delivered in vivo, for example, as part of a polypeptide or complexed with another macromolecule, with or without adjuvant via the intravenous, subcutaneous, intranasal, intramuscular or intraperitoneal route of delivery.

Various other techniques can be used, including the following. Paglia et al. (1996) J. Exp. Med. 183:317-322 has shown that APC incubated with whole protein in vitro are recognized by MHC class I-restricted CTLs, and that immunization of animals with these APCs led to the development of antigen-specific CTLs in vivo. In addition, several different techniques have been described which lead to the expression of antigen in the cytosol of APCs, such as DCs. These include (1) the introduction into the APCs of RNA isolated from tumor cells, (2) infection of APCs with recombinant vectors to induce endogenous expression of antigen, and (3) introduction of tumor antigen into the DC cytosol using liposomes. (See Boczkowski et al. (1996) J. Exp. Med. 184:465-472; Rouse et al. (1994) J. Virol. 68:5685-5689; and Nair et al. (1992) J. Exp. Med. 175:609-612).

Another method which can be used is termed “painting.” It has been demonstrated that glycosyl-phosphotidylinositol (GPI)-modified proteins possess the ability to reincorporate themselves back into cell membranes after purification. Hirose et al. (1995) Methods Enzymol. 250:582-614; Medof et al., (1984) J. Exp. Med. 160:1558-1578; Medof (1996) FASEB J. 10:574-586; and Huang et al. (1994) Immunity 1:607-613 have exploited this property in order to create APCs of specific composition for the presentation of antigen to CTLs. They devised expression vectors for β2-microglobulin and the HLA-A2.1 allele. The proteins were expressed in Schneider S2 Drosophila melanogaster cells, known to support GPI-modification. After purification, the proteins could be incubated together with a purified antigenic peptides which resulted in a trimolecular complex capable of efficiently inserting itself into the membranes of autologous cells. In essence, these protein mixtures were used to “paint” the APC surface, conferring the ability to stimulate a CTL clone that was specific for the antigenic peptide. Cell coating was shown to occur rapidly and to be protein concentration dependent. This method of generating APCs bypasses the need for gene transfer into the APC and permits control of antigenic peptide densities at the cell surfaces.

Immune Effector Cells

The present invention makes use of the above-described compositions including APCs, to stimulate production of an enriched population of antigen-specific immune effector cells. Accordingly, the present invention provides a population of cells enriched in educated, antigen-specific immune effector cells, specific for an antigenic peptide of the invention. These cells can cross-react with (bind specifically to) antigenic determinants (epitopes) on natural (endogenous) antigens. In some embodiments, the natural antigen is on the surface of tumor cells and the educated, antigen-specific immune effector cells of the invention suppress growth of the tumor cells. When APCs are used, the antigen-specific immune effector cells are expanded at the expense of the APCs, which die in the culture. The process by which naive immune effector cells become educated by other cells is described essentially in Coulie (1997) Molec. Med. Today 3:261-268.

The APCs prepared as described above are mixed with naive immune effector cells. Preferably, the cells may be cultured in the presence of a cytokine, for example IL2. Because dendritic cells secrete potent immunostimulatory cytokines, such as IL-12, it may not be necessary to add supplemental cytokines during the first and successive rounds of expansion. In any event, the culture conditions are such that the antigen-specific immune effector cells expand (i.e., proliferate) at a much higher rate than the APCs. Multiple infusions of APCs and optional cytokines can be performed to further expand the population of antigen-specific cells.

In one embodiment, the immune effector cells are T cells. In a separate embodiment, the immune effector cells can be genetically modified by transduction with a transgene coding for example, IL-2, IL-11 or IL-13. Methods for introducing transgenes in vitro, ex vivo and in vivo are well known in the art. See Sambrook, et al. (1989) supra.

An effector cell population suitable for use in the methods of the present invention can be autogeneic or allogeneic, preferably autogeneic. When effector cells are allogeneic, preferably the cells are depleted of alloreactive cells before use. This can be accomplished by any known means, including, for example, by mixing the allogeneic effector cells and a recipient cell population and incubating them for a suitable time, then depleting CD69⁺ cells, or inactivating alloreactive cells, or inducing anergy in the alloreactive cell population.

Hybrid immune effector cells can also be used. Immune effector cell hybrids are known in the art and have been described in various publications. See, for example, International Patent Application Nos. WO 98/46785 and WO 95/16775.

The effector cell population can comprise unseparated cells, i.e., a mixed population, for example, a PBMC population, whole blood, and the like. The effector cell population can be manipulated by positive selection based on expression of cell surface markers, negative selection based on expression of cell surface markers, stimulation with one or more antigens in vitro or in vivo, treatment with one or more biological modifiers in vitro or in vivo, subtractive stimulation with one or more antigens or biological modifiers, or a combination of any or all of these.

Effector cells can obtained from a variety of sources, including but not limited to, PBMC, whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include an immunized donor, a non-immunized (naïve) donor, treated or untreated donors. A “treated” donor is one that has been exposed to one or more biological modifiers. An “untreated” donor has not been exposed to one or more biological modifiers.

Methods of extracting and culturing effector cells are well known. For example, effector cells can be obtained by leukapheresis, mechanical apheresis using a continuous flow cell separator. For example, lymphocytes and monocytes can be isolated from the buffy coat by any known method, including, but not limited to, separation over Ficoll-Hypaque™ gradient, separation over a Percoll gradient, or elutriation. The concentration of Ficoll-Hypaque™ can be adjusted to obtain the desired population, for example, a population enriched in T cells. Other methods based on affinity are known and can be used. These include, for example, fluorescence-activated cell sorting (FACS), cell adhesion, magnetic bead separation, and the like. Affinity-based methods may utilize antibodies, or portions thereof, which are specific for cell-surface markers and which are available from a variety of commercial sources, including, the American Type Culture Collection (Manassas, Md.). Affinity-based methods can alternatively utilize ligands or ligand analogs, of cell surface receptors.

The effector cell population can be subjected to one or more separation protocols based on the expression of cell surface markers. For example, the cells can be subjected to positive selection on the basis of expression of one or more cell surface polypeptides, including, but not limited to, “cluster of differentiation” cell surface markers such as CD2, CD3, CD4, CD8, TCR, CD45, CD45RO, CD45RA, CD11b, CD26, CD27, CD28, CD29, CD30, CD31, CD40L; other markers associated with lymphocyte activation, such as the lymphocyte activation gene 3 product (LAG3), signaling lymphocyte activation molecule (SLAM), T1/ST2; chemokine receptors such as CCR3, CCR4, CXCR3, CCR5; homing receptors such as CD62L, CD44, CLA, CD146, a4b7, aEb7; activation markers such as CD25, CD69 and OX40; and lipoglycans presented by CD1. The effector cell population can be subjected to negative selection for depletion of non-T cells and/or particular T cell subsets. Negative selection can be performed on the basis of cell surface expression of a variety of molecules, including, but not limited to, B cell markers such as CD19, and CD20; monocyte marker CD14; the NK cell marker CD56.

An effector cell population can be manipulated by exposure, in vivo or in vitro, to one or more biological modifiers. Suitable biological modifiers include, but are not limited to, cytokines such as IL-2, IL-4, IL-10, TNF-α, IL-12, IFN-γ; non-specific modifiers such as phytohemagglutinin (PHA), phorbol esters such as phorbol myristate acetate (PMA), concanavalin-A, and ionomycin; antibodies specific for cell surface markers, such as anti-CD2, anti-CD3, anti-IL2 receptor, anti-CD28; chemokines, including, for example, lymphotactin. The biological modifiers can be native factors obtained from natural sources, factors produced by recombinant DNA technology, chemically synthesized polypeptides or other molecules, or any derivative having the functional activity of the native factor. If more than one biological modifier is used, the exposure can be simultaneous or sequential.

The present invention provides compositions comprising immune effector cells, which may be T cells, enriched in antigen-specific cells. By “enriched” is meant that a cell population is at least about 50-fold, more preferably at least about 500-fold, and even more preferably at least about 5000-fold or more enriched from an original naive cell population. The proportion of the enriched cell population which comprises antigen-specific cells can vary substantially, from less than 10% up to 100% antigen-specific cells. If the cell population comprises at least 50%, preferably at least 70%, more preferably at least 80%, and even more preferably at least 90%, antigen-specific immune effector cells, specific for a peptide of the invention, then the population is said to be “substantially pure.” The percentage which are antigen-specific can readily be determined, for example, by a ³H-thymidine uptake assay in which the effector cell population (for example, a T-cell population) is challenged by an antigen-presenting matrix presenting an antigenic peptide of the invention.

Compositions of the Invention

This invention also provides compositions containing any of the above-mentioned peptides, polypeptides, polynucleotides, antigen-presenting matrices, vectors, cells, antibodies and fragments thereof, and an acceptable solid or liquid carrier. When the compositions are used pharmaceutically, they are combined with a “pharmaceutically acceptable carrier” for diagnostic and therapeutic use. These compositions also can be used for the preparation of medicaments for the diagnostic and immunomodulatory methods of the invention.

Methods of the Invention

The present invention provides diagnostic and immunomodulatory methods using peptides, polynucleotides, and host cells (including APCs and educated immune effector cells), i.e., immunomodulatory agents, of the invention.

Diagnostic Methods

The present invention provides diagnostic methods using altered peptide ligands of the invention. The methods can be used to detect the presence of an antigen-specific CD4⁺ or CD8⁺ T cell which binds the altered peptide ligands of the invention. Such a T cell is expected to also bind a natural counterpart to the synthetic peptide.

The diagnostic methods of the invention include: (1) assays to predict the efficacy of an altered peptide ligand of the invention; (2) assays to determine the precursor frequency (i.e., the presence and number of) of immune effector cells specific for an altered peptide ligand and/or its natural counterpart; and (3) assays to determine the efficacy of an altered peptide ligand once it has been used in an immunomodulatory method of the invention.

Diagnostic methods of the invention are generally carried out under suitable conditions and for a sufficient time to allow specific binding to occur between an altered peptide ligand or its natural counterpart and an immune effector molecule, such as a TCR, on the surface of an immune effector cell, such as a CD4+ or CD8+ T cell. “Suitable conditions” and “sufficient time” are generally conditions and times suitable for specific binding. Suitable conditions occur between about 4° C. and about 40° C., preferably between about 4° C. and about 37° C., in a buffered solution, and within a pH range of between 5 and 9. A variety of buffered solutions are known in the art, can be used in the diagnostic methods of this invention, and include, but are not limited to, phosphate-buffered saline. Sufficient time for binding and response will generally be between about 1 second and about 24 hours after exposure of the sample to the convergent antigenic peptide ligand.

In some embodiments, the invention provides diagnostic assays to predict the efficacy of an altered peptide ligand. In some of these embodiments, defined T cell epitopes are used to clinically characterize tumors and viral pathogens in order to determine in advance the predicted efficacy of an in vivo vaccine trial. This can be achieved by a simple proliferation assay of a patient's peripheral blood mononuclear cells using defined T cell epitopes as stimulators. Altered peptide ligands that elicit a response are viable vaccine candidates for that patient.

In other embodiments, assays are provided to determine the precursor frequency (i.e., the presence and number of) of resting (naive) immune effector cells specific for an altered peptide ligand and/or its natural counterpart, and which therefore have the potential to become activated. In these embodiments, an antigen-presenting cell bearing on its surface a natural counterpart of an altered peptide ligand is used to detect the presence of immune effector cells in a biological sample which bind specifically to the natural epitope. A functional assay is used to determine (and quantitate) the antigen-specific immune effector cells. As an illustrative example, PBMCs are isolated from a subject with a tumor. A sample of these PBMCs is cultured together for a suitable time with the tumor cells from the same subject. A second sample of these PBMCs is cultured together for a suitable time with surrogate APCs pulsed with an appropriate CAP. Both tumor cells and surrogate APCs are loaded with ⁵¹Cr. By comparing the amount of ⁵¹Cr release from the tumor cell and the antigen-pulsed surrogate APC, one can determine the precursor frequency of immune effector cells which are specific for tumor and the precursor frequency of immune effector cells which are specific for the altered peptide ligands or their corresponding wild-type antigenic peptide. Functional assays include, but are not limited to, immune effector cell proliferation, cytokine production, specific lysis of an APC.

In other embodiments, the efficacy of an immunomodulatory method, including immunomodulatory methods of the invention, in modulating an immune response to an altered peptide ligand of the invention and/or its natural counterpart, can be tested using diagnostic assays of the invention. These diagnostic assays are also useful to assess or monitor the efficacy of an immunotherapeutic agent. In some of these embodiments, the method allows detection of immune effector cells, which may be activated CD4⁺ or CD8⁺ T cells, which have become activated or anergized as a result of exposure to altered peptide ligands of the invention. A sample containing cells from a subject can be tested for the presence of CD4⁺ or CD8⁺ T cells which have become activated or anergized as a result of binding to a given altered peptide ligand of the invention.

The agents provided herein as effective for their intended purpose can be administered to subjects having a disease to be treated with an immunomodulatory method of the invention or to individuals susceptible to or at risk of developing such a disease. When the agent is administered to a subject such as a mouse, a rat or a human patient, the agent can be added to a pharmaceutically acceptable carrier and systemically or topically administered to the subject. Therapeutic amounts can be empirically determined and will vary with the pathology or condition being treated, the subject being treated and the efficacy and toxicity of the therapy.

The amount of a peptide or immune effector cell of the invention will vary depending, in part, on its intended effect, and is ultimately at the discretion of the medical or veterinary practitioner. The factors to be considered include the condition being treated, the route of administration, and nature of the formulation, the mammal's body weight, surface area, age, and general condition and the particular peptide to be administered. A suitable effective dose of peptides of the invention generally lies in the range of from about 0.0001 μmol/kg to about 1000 μmol/kg bodyweight. The total dose may be given as a single dose or multiple doses, e.g., two to six times per day. For example, for a 75 kg mammal (e.g., a human) the dose range would be about 2.25 μmol/kg/day and a typical dose could be about 100 μmol of peptide. If discrete multiple doses are indicated treatment might typically be 25 μmol of a peptide of the invention given up to 4 times per day. In an alternative administrative regimen, peptides of the invention may be given on alternate days or even once or twice a week. A suitable effective dose of an immune effector cell of the invention generally lies in the range of from about 10² to about 10⁹ cells per administration. Cells can be administered once, followed by monitoring of the clinical response, such as diminution of disease symptoms or tumor mass. Administration may be repeated on a monthly basis, for example, or as appropriate. Those skilled in the art will appreciate that an appropriate administrative regimen would be at the discretion of the physician or veterinary practitioner.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

More particularly, an agent of the present invention also referred to herein as the active ingredient, may be administered for therapy by any suitable route including nasal, topical (including transdermal, aerosol, buccal and sublingual), parenteral (including subcutaneous, intramuscular, intravenous and intradermal) and pulmonary. It will also be appreciated that the preferred route will vary with the condition and age of the recipient, and the disease or condition being treated.

Methods for Determining Vβ Repertoire

Various methods are know in the art to determine the sequence of the Vβ region of the T cells. These methods use modification of the polymerase chain reaction (PCR), cell staining and flow cytometery. Such methods are described in Lee, K-H et al. (1998) J. Immun. 161:4183-4194; Blattman, J. N., et al. (2000) J. Immun. 165: 6081-6090; Ben-Nun, A. et al. (1991) PNAS 88:2466-2470; Henwood, J. et al. (1995) Human Immun. 42:301-306 and McMahan and Fink (2000) J. Immun. 165:6902-6907.

Vaccines for Cancer Treatment and Prevention

In one embodiment, immunomodulatory methods of the present invention comprise vaccines for cancer treatment. Cancer cells contain many new antigens potentially recognizable by the immune system. Given the speed with which epitopes can be identified, custom anticancer vaccines can be generated for affected individuals by isolating TILs from patients with solid tumors, determining their MHC restriction, and assaying these CTLs against the appropriate library for reactive epitopes. These vaccines will be both treatments for affected individuals as well as preventive therapy against recurrence (or establishment of the disease in patients which present with a familial genetic predisposition to it). Inoculation of individuals who have never had the cancer is expected to be quite successful as preventive therapy, even though a tumor antigen-specific CTL response has not yet been elicited, because in most cases high affinity peptides seem to be immunogenic suggesting that holes in the functional T cell repertoire, if they exist, may be relatively rare. Sette et al. (1994) J. Immunol., 153:5586-5592. In mice, vaccination with appropriate epitopes not only eliminates established tumors but also protects against tumor re-establishment after inoculation with otherwise lethal doses of tumor cells. Bystryn et al. (1993) supra.

Recent advances in vaccine adjuvants provide effective means of administering peptides so that they impact maximally on the immune system. Del-Giudice (1994) Experientia 50:1061-1066. These peptide vaccines will be of great value in treating metastatic tumors that are generally unresponsive to conventional therapies. Tumors arising from the homozygous deletion of recessive oncogenes are less susceptible to elimination by a humoral (antibody) response and would thus be treated more effectively by eliciting a cellular, CTL response.

Vaccines for Diseases Caused by Pathogenic Organisms

Altered peptide ligands of the present invention are also useful in methods to induce (or increase, or enhance) an immune response to a pathogenic organism. These include pathogenic viruses, bacteria, and protozoans.

Viral infections are ideal candidates for immunotherapy. Immunological responses to viral pathogens are sometimes ineffective as in the case of the lentiviruses such as HIV which causes AIDS. The high rates of spontaneous mutation make these viruses elusive to the immune system. However, a saturating profile of CTL epitopes presented on infected cells will identify shared antigens among different serotypes in essential genes that are largely intolerant to mutation which would allow the design of more effective vaccines.

Adoptive Immunotherapy Methods

The expanded populations of antigen-specific immune effector cells and APCs of the present invention find use in adoptive immunotherapy regimes and as vaccines.

Adoptive immunotherapy methods involve, in one aspect, administering to a subject a substantially pure population of educated, antigen-specific immune effector cells made by culturing naive immune effector cells with APCs as described above. In some embodiments, the APCs are dendritic cells.

In one embodiment, the adoptive immunotherapy methods described herein are autologous. In this case, the APCs are made using parental cells isolated from a single subject. The expanded population also employs T cells isolated from that subject. Finally, the expanded population of antigen-specific cells is administered to the same patient.

In a further embodiment, APCs or immune effector cells are administered with an effective amount of a stimulatory cytokine, such as IL-2 or a co-stimulatory molecule.

The following examples are intended to illustrate, but not limited to the present invention.

### EXPERIMENTAL EXAMPLES

A series of assays were conducted in which the native melanoma antigen gp100 and altered peptide ligands educate T cells obtained from normal (healthy) donors (of a designated HLA type). The educated T cells were then assessed for their ability to recognize and lyse both target cells displaying the “educating” altered peptide as well as target cells displaying the native peptide.

The results showed that T cells educated with the native gp100 peptide ligand were generally inefficient in their ability to lyse targets displaying the native antigen, whereas the T cells educated with the altered gp100 peptide ligands were able to lyse targets displaying the altered ligands and able to lyse targets displaying the native ligand.

### MATERIALS AND METHODS

Cell lines and reagents. TIL1520, TIL620-10, and TIL1235 were generously provided by M. Nishimura (NIH, Surgery branch). This T cell clone was maintained in AIM V medium (Gibco, Carlsbad, Calif.) supplemented with 10% human AB serum (Sigma, St. Louis, Mo.), Penicillin, streptomycin, and 6000 IU/ml human recombinant IL-2 (Proleukin, Chiron, Emeryville, Calif.). A549 and T2 cells were obtained through ATCC and maintained in DMEM/10% FBS and RPMI1640/10% FBS (JRH Bioscience, Lenexa, Tex.), respectively.

Peptide sequencing. Peptide sequencing was performed by Edman degradation.

Library screening. Screens were all performed identically, employing a routine ⁵¹Cr-release microcytotoxicity assay the following modifications. 2 μl released peptide was added to V bottom 96-well plates and T2 cells were added at a density of 1000 cells/well in a total volume of 100 μl/well and incubated at 37° C./5% CO₂ for 60 minutes. 1000 T cells in 100 μl RPMI1640/10% human AB serum was then added to each well and the plates were returned to the incubator for 4 hours. Supernatant was harvested (25 μl) from each well and the amount of released ⁵¹Cr quantitated using a Wallach TriLux MicroBeta plate counter (Turleu, Finland). Spontaeous ⁵¹Cr release was measured in the absence of effector T cells and total ⁵¹Cr release was measured by lysing the cells with 0.1% Triton X-100. Percent specific lysis was calculated according to the following formula:

$$100 \times \frac{( experimental - spontaneous )}{( total - spontaneous )}$$

In vitro T cell education studies.

Normal donor aphoresis products were obtained from Dana-Farber Cancer Research Institute (Boston, Mass.). PBMC were isolated by centrifugation over Ficoll (Nycomed, Oslo, Norway). CD8⁺ T cells were isolated using magnetic beads (Dynal, Oslo, Norway) according to the manufacturers instructions. To generate autologous dendritic cells, monocytes were isolated from the PBMC and treated with GM-CSF (Immunex, Seattle, Wash.) and IL-4 (PeproTech, London, England) for 6 days as previously described. One day prior to establishing the T cell/DC cocultures, the DCs were pulsed with peptides (10 μg/ml) overnight followed by the addition of the previously isolated CD8+ T cells at a T:DC of 10:1. Cultures were restimulated with peptide-pulsed DCs. IL-2 (50 IU/ml) was introduced on day 8 and added every 3-4 days as needed. The bulk cultures were assayed one week after the fifth restimulation. Peptide-pulsed T2 cell targets were prepared as described above and adenovirus-infected target cells were allowed to incubate with the viruses for 48 hours at the indicated MOI prior to being used in the CTL assay. All cultures were assayed in quadruplicate using 1e+4 ⁵¹Cr-labeled target cells at the indicated E:T for 16 hours.

Altered peptides react specifically with native epitope-specific CTL.

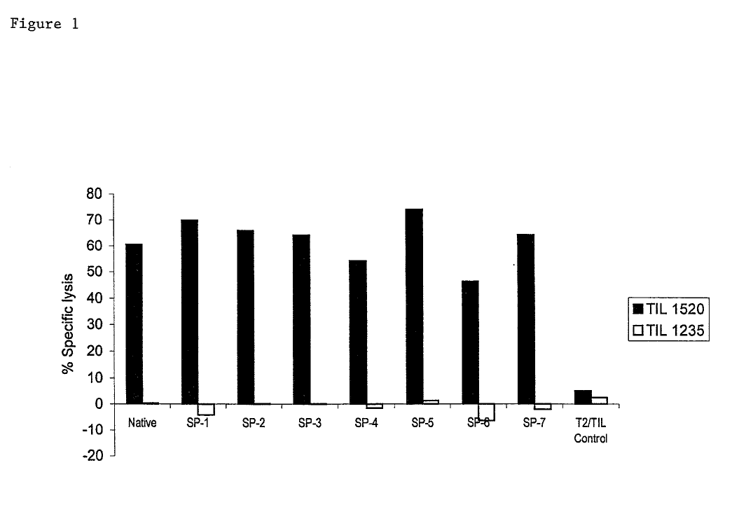

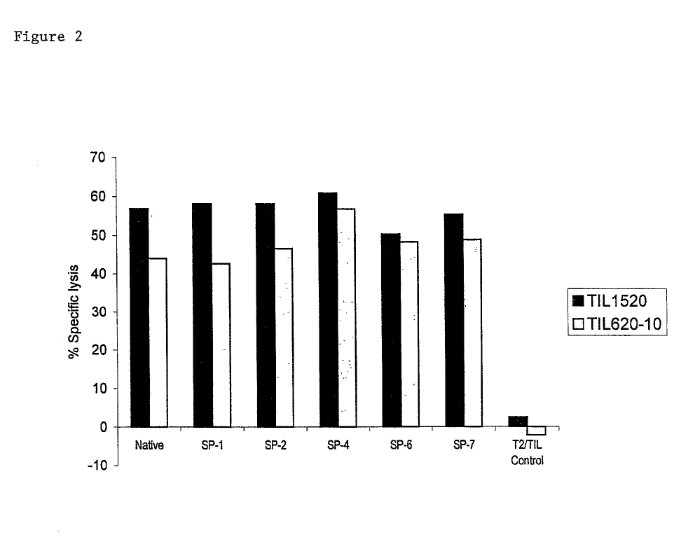

Since some of the peptides are divergent from the native epitope that reactivity with TIL1520 was confirmed. The reactivity of some of these peptides in a ⁵¹Cr-release assay with the T cell clone used in the screen (TIL1520) or an irrelevant clone (TIL1235). FIG. 1 shows the result of this assay. Next screening of the altered peptides for the ability to react with an independently derived gp100₂₀₉₋₂₁₇-specific TIL population (TIL620-10) was performed. To this end, a subset of the altered peptides were chosen for their sequence diversity, were tested for reactivity with either TIL1520 or TIL620-10 in a ⁵¹ Cr-release assay. These results are shown in FIG. 2 and indicate that the peptides react equally well with TIL620-10, implying that even the distantly related epitope mimics are functionally similar to the native epitope in this assay.

Altered Peptide Ligands are Potent Immunogens.

In order to characterize altered peptide ligands of the human melanoma antigen gp100, their relative abilities to educate normal donor HLA-A2⁺ T cells in vitro were tested. These in vitro T cell education studies were designed to test the ability of the altered peptide ligands to expand and sensitize T cells to lyse targets presenting the native epitope or targets presenting the peptides themselves.

Altered peptides give rise to T cells that recognize the native epitope. Normal donor T cells were educated in vitro with altered peptide- or wild-type gp100₂₀₉₋₂₁₇ peptide-pulsed autologous dendritic cells. After 5 weekly stimulations, bulk T cell cultures were tested for their abilities to lyse ⁵¹Cr-labeled T2 cells pulsed with the native peptide. For all assays, total peptide concentration was kept constant so that peptide combinations contained ⅔ less of each individual peptide compared to when they were used separately. All data points represent the average of 4 replicates and background lysis, as determined using T2 cells pulsed with equivalent amounts of DMSO containing no peptide, was subtracted out.

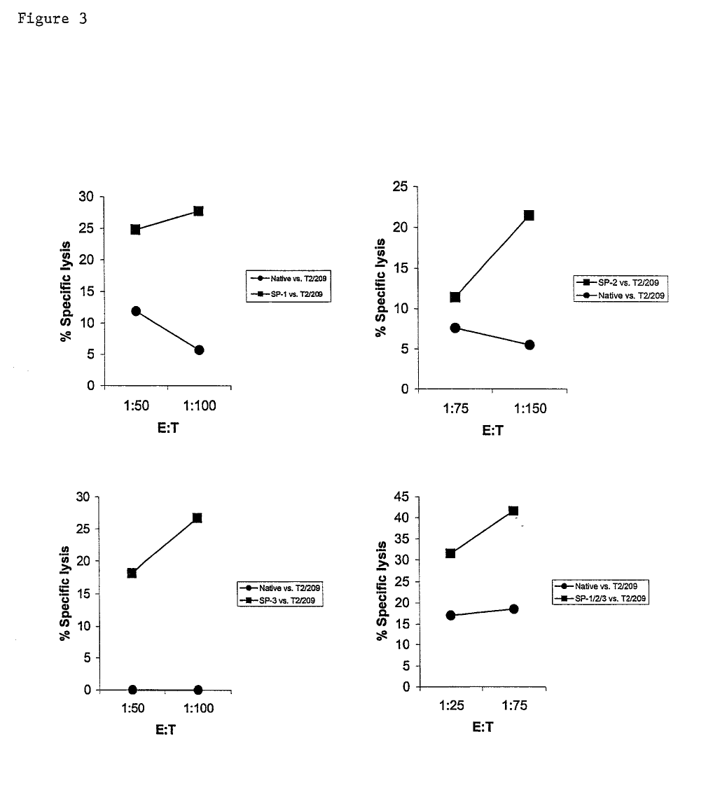

Results showed that the native epitope was relatively poor at eliciting reactive T cells in this assay, whereas the altered peptide ligands were capable of eliciting responses even in those individuals that responded poorly or not at all to the wild-type peptide. See FIG. 3. When these studies were extended to include a total of 20 normal donors, it was noted that, while no single altered peptide ligand was immunogenic in every donor, there were differential responses suggesting that the T cells each altered peptide ligand preferentially stimulated represented different populations, perhaps with different donor-dependent precursor frequencies. Analysis of T cell receptor Vβ usage within the in vitro educated bulk cultures of normal donor T cells by PCR analysis confirms this finding. This was further supported by the marked increase in population coverage observed when the peptides were used in combination with one another.

Altered peptide-educated T cells demonstrate exquisite specificity for the naturally processed and presented native epitope.

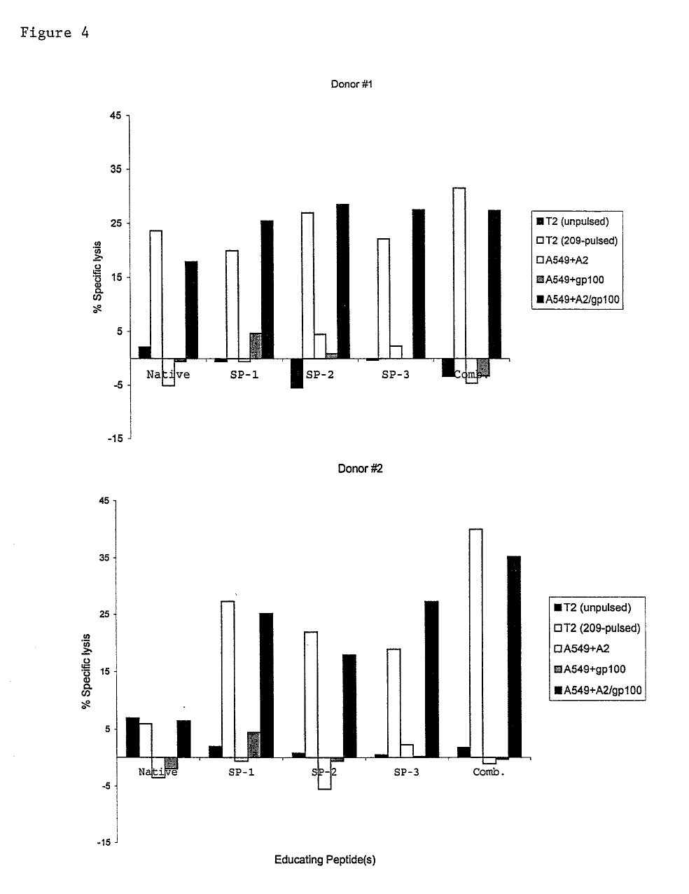

Given the divergence of the altered peptide sequences from the native epitope, the specificity of the T cells educated with these peptides was determind. To this end, in vitro-educated T cells were tested for reactivity to a human lung tumor cell line (A549) which is both HLA-A2⁻ and gp100⁻ See FIG. 4.

Altered peptide-educated normal donor T cells lyse targets expressing wild-type gp100 in an HLA-A2-specific manner.

Normal donor T cells were educated in vitro with altered peptide- or wild-type gp100₂₀₉-217 peptide-pulsed autologous dendritic cells. After 5 weekly stimulations bulk T cell cultures were tested for their abilities to lyse the lung cancer cell line A549 infected with adenoviruses expressing HLA-A2 and/or gp100 wild-type protein. The cells were infected with the viruses at an MOI of 25 for 48 hours and labeled with ⁵¹Cr. The in vitro-educated bulk T cell cultures were added at an E:T of 75:1, using 1e+4 targets. Percent specific lysis was calculated as described above.

When the cell line was converted to HLA-A2⁺ or gp100⁺ by recombinant adenovirus infection, the altered peptide-educated normal donor T cells still did not lyse them. However, when the cell line was doubly infected to express both HLA-A2 and gp100, the T cells educated with any of the altered peptides tested in these experiments lysed the cells. These data demonstrate that altered peptides can produce functionally indistinguishable HLA-restricted, antigen-specific responses and that naturally processed and presented peptide from the native antigen can render tumor cells susceptible to lysis by these effectors.

Although the foregoing invention has been described in some detail by way of illustration and example for purposes of clarity of understanding, it will be apparent to those skilled in the art that certain changes and modifications will be practiced. Therefore, the description and examples should not be construed as limiting the scope of the invention, which is delineated by the appended claims.

What is claimed is:

1. A method to select altered peptide species for administration to a subject, said subject presenting a native ligand for which activation of an immune response against said native ligand is desired, comprising the steps of:
a. identifying a plurality of altered peptide species capable of eliciting an immune response to said native ligand;
b. selecting at least two altered peptide species wherein each altered peptide activates a population of T cells having a distinct T cell receptor Vβ recombination.

2. The method of claim 1, further comprising the step of administering said selected altered peptide species to said subject.

3. A method to select altered peptide species for administration to a member of a population of subjects having a given HLA-type, said population presenting a native ligand for which activation of an immune response against said native ligand is desired, comprising the steps of:
a. identifying a plurality of altered peptide species capable of eliciting an immune response to said native ligand; and
b. selecting two or more of said altered peptide species, wherein each of said altered peptide species activates a T cell having a distinct T cell receptor Vβ recombination in a sample representative of said population.

4. The method of claim 3, further comprising the step of administering said selected altered peptide species to said member.

5. The method of claim 3, wherein said HLA-type is HLA-A2.

6. The method of claims 1 or 3, comprising from at least 2 to 6 different altered peptide species.

7. The method of claims 1 or 3, comprising at least 3 different altered peptide species.

8. The method of claims 1 or 3, wherein at least one altered peptide species is covalently linked to one or more amino acids naturally contiguous to said native human ligand.

9. The method of claims 1 or 3, wherein said native ligand is a mammalian tumor epitope.

10. The method of claims 1 or 3, wherein said native ligand is a human viral antigen.

11. The method of claims 1 or 3, further comprising the step of formulating said selected altered peptides in a pharmaceutically acceptable carrier suitable for administration to humans.

12. A composition comprising multiple peptide ligand species directed at a single native ligand, wherein at least two altered peptide species activate a different T cell clone from each other and the T cell receptor Vβ recombination of each of said activated T cell clones is different.

13. A composition comprising two or more altered peptide species, wherein each of said two or more species is characterized by an ability to activate a different subpopulation of cytotoxic T lymphocytes (CTLs) against the same native ligand.

14. The composition of claims 12 or 13, comprising from at least 2 to 6 different peptide species.

15. The composition of claims 12 or 13, comprising at least 3 different peptide species.

16. The composition of claims 12 or 13, wherein at least one peptide species is covalently linked to one or more amino acids naturally contiguous to said native human ligand.

17. The composition of claim 13, wherein said altered peptide species activate said different subpopulations in one member of a selected population of subjects

18. The composition of claim 13, wherein said altered peptide species activate a different subpopulation in two or more members of said population of subjects.

19. The composition of claims 12 or 13, wherein said native ligand is a mammalian tumor epitope.

20. The composition of claims 12 or 13, wherein said native ligand is a human viral antigen.

21. The composition of claims 12 or 13, further comprising a pharmaceutically acceptable carrier.

22. A kit comprising:
a. multiple peptide species directed at a single native ligand, wherein
i. a first peptide species activates a first T cell,
ii. a second peptide species activates a second T cell, and the T cell receptor Vβ recombination of said first T cell is different from the T cell receptor Vβ recombination of said second T cell; and
b. instructions for the co-administration of each of said peptides.

23. The kit of claim 22, wherein said selected peptide species are packaged in combination.

24. The kit of claim 22, further comprising instructions to identify subjects who exhibit a positive therapeutic response to the administration of said multiple peptide ligand species.

25. A method comprising the steps of:
a. identifying a plurality of altered peptide species characterized by an ability to elicit an immune response against the same native ligand;
b. determining the T cell receptor Vβ recombination profile of the T cell population activated by each of said identified altered peptide species in a plurality of test subjects; and
selecting at least two or more altered peptide species wherein each of said altered peptide species activates T cell populations having distinct T cell receptor Vβ recombinations in a majority of said test subjects.

26. The method of claim 25, wherein at least one of said selected altered peptide species is more than said native antigen.

27. The method of claim 25, further comprising the step of administering said selected altered peptide species to a subject having said native antigen.

28. The method of claim 25, further comprising the step of packaging said selected altered peptide species in a form suitable for administration to a subject.

29. The method of claim 26, wherein said selected altered peptide species are packaged together.
